(19) United States
(12) Patent Application Publication
(10) Pub. No.: US 2022/0093285 A1
(43) Pub. Date: Mar. 24, 2022

(54) ADAPTIVE FOCUS COLLIMATION OF X-RAY BEAMS

(71) Applicant: RefleXion Medical, Inc., Hayward, CA (US)

(72) Inventor: Jon William BURNS, Oakland, CA (US)

(21) Appl. No.: 17/543,550

(22) Filed: Dec. 6, 2021

Related U.S. Application Data
(63) Continuation of application No. PCT/US2020/038218, filed on Jun. 17, 2020.
(60) Provisional application No. 62/862,510, filed on Jun. 17, 2019.

(51) Int. Cl. G21K 1/04; A61N 5/10

(52) U.S. Cl. CPC G21K 1/046; A61N 5/1077

(57) ABSTRACT

In some embodiments, a radiation beam-shaping assembly comprises a pair of tiltable jaws. Each jaw of the pair of tiltable jaws is laterally movable relative to a radiation beam path.

### CROSS-REFERENCE TO RELATED APPLICATIONS

This application is a continuation of International Patent Application No. PCT/US2020/038218, entitled “Adaptive Focus Collimation of X-Ray Beams,” filed on Jun. 17, 2020, which claims priority to U.S. Provisional Patent Application No. 62/862,510, entitled “Adaptive Focus Collimation of X-Ray Beams,” filed on Jun. 17, 2019, the disclosure of which is incorporated by reference herein in its entirety.

### BACKGROUND

Radiation therapy involves delivering radiation to a tumor. In some radiation therapy systems, a radiation source mounted on a gantry rotates around a patient on a table or couch, and directs radiation toward the patient's tumor(s) from various firing positions around the patient. As the radiation source rotates around the patient, the patient table or couch may be moved in a direction that is parallel to the axis of rotation of the radiation source. In this manner, radiation may be applied to the patient's tumor(s) from various gantry angles and at various patient table or couch positions, based on images of the patient and the tumor(s) generated by various imaging modalities in advance of the treatment session.

Tumors may vary in size, shape, and location within a patient. Furthermore, tumors are often asymmetrical and include portions varying in distance from a patient surface. Applying a radiation beam to a target region of a patient may result in the exposure of healthy tissue to radiation and/or result in portions of a tumor not receiving sufficient radiation to effectively treat the tumor. Thus, there is a desire for radiation beam-shaping assemblies that may better target tumors of varying shape, size, and location within a patient while limiting irradiation of healthy tissue.

### BRIEF SUMMARY

Disclosed herein are radiation beam-shaping assemblies for radiation therapy systems. In some embodiments, a radiation beam-shaping assembly comprises a pair of tiltable jaws. Each jaw of the pair of tiltable jaws is laterally movable relative to a radiation beam path.

A first jaw of the pair of tiltable jaws may be movable in concert with a second jaw of the pair of tiltable jaws. Lateral movement of each jaw of the pair of tiltable upper jaws relative to the radiation beam path may adjust the size of an aperture defined between the pair of tiltable upper jaws such that a radiation beam field is adjusted.

A first actuator and a second actuator may be coupled to a first jaw of the pair of tiltable jaws. A lateral movement of the first jaw may be based, at least in part, on a combined rotation of the first jaw by the first actuator and counter-rotation of the first jaw by the second actuator. The lateral movement of each jaw of the pair of tiltable jaws may be linear. The lateral movement of each jaw of the pair of tiltable jaws may be curvilinear.

The pair of tiltable jaws may be a pair of tiltable upper jaws and the radiation beam-shaping assembly may further comprise a pair of tiltable lower jaws. Each jaw of the pair of tiltable lower jaws may be laterally movable relative to a radiation beam path. A first jaw of the pair of tiltable upper jaws may be movable in concert with a first jaw of the pair of tiltable lower jaws. A second jaw of the pair of tiltable upper jaws may be movable in concert with a second jaw of the pair of tiltable lower jaws. A first jaw of the pair of tiltable upper jaws and a first jaw of the pair of tiltable lower jaws may be mounted to a first carriage and a second jaw of the pair of tiltable upper jaws and a second jaw of the pair of tiltable lower jaws may be mounted to a second carriage.

The radiation beam-shaping assembly may include a multi-leaf collimator assembly located between the pair of tiltable upper jaws and the pair of tiltable lower jaws. Additionally or alternatively, the radiation beam-shaping assembly may include a third pair of tiltable jaws. Each jaw of the third pair of tiltable jaws may be laterally movable relative to the radiation beam path.

### BRIEF DESCRIPTION OF THE DRAWINGS

FIG. 1 is a schematic illustration of a radiation beam-shaping assembly, according to an embodiment.

FIG. 2A is a schematic representation of a radiation beam-shaping assembly in a first configuration, according to an embodiment.

FIG. 2B is a schematic representation of the radiation beam-shaping assembly of FIG. 2A in a second configuration.

FIGS. 2C-2H are schematic representations of jaws of the radiation beam-shaping assembly of FIG. 2A in various tilt angle orientations.

FIG. 3 is a perspective view of a portion of a radiation beam-shaping assembly, according to an embodiment.

FIGS. 4A-4K are various illustrative depictions of a radiation beam-shaping assembly in various configurations, according to an embodiment.

FIGS. 5A and 5B are schematic illustrations of an iris diaphragm in a first configuration and a second configuration, respectively, according to an embodiment.

### DETAILED DESCRIPTION

Disclosed herein are radiation beam-shaping assemblies for radiation therapy systems. In some embodiments, a radiation beam-shaping assembly comprises a pair of tiltable jaws. Each jaw of the pair of tiltable jaws is laterally movable relative to a radiation beam path. As used herein, “jaw” may refer to any beam-limiting element, beam-defining element, beam-shaping element, collimation element, or any other radiation-attenuating or radiation-obstructing element or structure that may be included in a device that is used to define a shape of a radiation beam. For example, a beam-shaping (i.e., beam-limiting, beam-defining, collimation, etc.) device may comprise one or more jaws, beam-limiting elements, beam-defining elements, beam-shaping elements, and/or collimation elements, alone or in combination, to alter the characteristics of a radiation beam/field. Illustrative variations of beam-shaping devices comprising one or more jaws and how such devices are configured to adjust one or more properties of radiation beams/fields are provided herein.

The focus and/or characteristics of a radiation field may be determined by the orientation and position of each of the jaws relative to each other and/or a central axis of the radiation beam. For example, the radiation field size, shape, and sharpness of the edge of the radiation field and/or penumbra may be tuned by adjusting the orientation and position of the jaws. In some variations, a first jaw and a second jaw of the pair of tiltable jaws may be titled or rotated, and/or laterally translated relative to a radiation beam (e.g., relative to a central axis of the radiation beam), which may determine the shape of the radiation beam and characteristics of a radiation field created by the radiation beam. The characteristics of the radiation field may include, for example, a size of a primary or central region of the radiation field (where the field intensity or fluence is homogenous), a size of a secondary or border region (e.g., penumbra) of the radiation field, penumbra sharpness or feathering (e.g., the field intensity or fluence gradient or rate of drop-off from the primary or central region), fluence, and/or energy delivery depth. The distance between the jaws in a pair of jaws may determine the size of the primary or central region of the radiation field, while the orientation of each of the jaws (e.g., tilt angle) may determine the size and/or gradient of the penumbra. These radiation field characteristics may be tuned according to the size and shape of a patient target region and the prescribed dose to the patient target region. For example, for a particular patient target region (e.g., a tumor), a clinician may prescribe a higher dose to a portion of the patient target region and a lower dose to another portion of the patient target region. In this example, a first, homogenous portion of a tumor (e.g., central portion) may be irradiated with a higher fluence than a second, heterogeneous portion of the tumor disposed adjacent to healthy tissue (e.g., peripheral portions of target region and surrounding regions), so as to reduce irradiation of the healthy tissue. One variation of a method to deliver different dose levels to different tumor areas may comprise aligning a primary/central region of a radiation field with a first portion of the patient target region and a secondary/penumbra region of the radiation field with a second portion of the patient target region. The portion of the patient target region that is aligned or co-localized with the primary region of the radiation field may receive a higher radiation fluence than the portion of the patient target region that aligned or co-localized with the penumbra.

Any of the radiation beam-shaping assemblies described herein comprising one or more pairs of tiltable jaws may be configured to shape a radiation beam such that the radiation field has a sharp and clearly defined radiation field boundary (e.g., with a relatively small penumbra region or fast fluence gradient or drop-off from the homogenous central region of the radiation field). This may be desirable for some patient target regions, for example, tumor regions having a sharp or distinct edge. Beam-shaping assemblies comprising tiltable jaws may also be configured to shape a radiation beam such that the radiation field has a wide penumbra region (e.g., with a relatively larger penumbra region or a more gradual fluence gradient or drop-off from the homogenous central region of the radiation field). This may be desirable for irradiating patient target regions having a complex edge shape and/or a poorly-defined boundary (e.g., due to the possible existence of microscopic tumor particles and/or structures). The orientation and/or position of tiltable jaws may be adjusted to have a smaller aperture (i.e., smaller distance between the jaws) for irradiating a relatively small target region (e.g., 1 cm or smaller) and may be adjusted to have a larger aperture (i.e., larger distance between the jaws) for irradiating a relatively larger target region (e.g., greater than about 1 cm). The orientation and/or position of the pair of tiltable jaws of any of the beam-shaping assemblies described herein may be adjusted to improve treatment (e.g., by adjusting characteristics of the energy transmitted) to the target region of the patient based on the size and shape of the target region (e.g., by altering the penumbra shape and size).

In some variations, the first jaw and the second jaw may be arranged at different locations relative to a radiation beam (e.g., on opposing sides of a central axis of the radiation beam extending from a radiation source) such that an aperture is defined between the first jaw and the second jaw. Characteristics of the radiation field may be based on the shape and size of the aperture defined between the first jaw and the second jaw. For example, a size (e.g., a width) of the radiation field may increase as the first jaw and the second jaw move apart and a size of the aperture defined by the first jaw and the second jaw is increased. The size (e.g., a width) of the radiation field may decrease as the first jaw and the second jaw move closer to each other and a size of the aperture defined by the first jaw and the second jaw is decreased. A shape of the aperture may be adjusted by tilting the first jaw and/or the second jaw relative to a central axis of the radiation beam (e.g., a central axis of the radiation beam extending from an aperture in a primary collimator to a system isocenter). Thus, a portion of the first jaw and/or the second jaw defining a boundary of the aperture (e.g., a beam-facing surface) may be rotated and/or pivoted such that the aperture boundary has a different shape. The shape of the aperture boundary (e.g., defined in part by an angle of the beam-facing surfaces of each jaw) may influence characteristics of the edge region of the radiation field. For example, the orientation of the first jaw and/or the second jaw may affect the size and fluence gradient of a penumbra region of a radiation field.

In some variations, a radiation beam-shaping assembly may include a controller in communication with one or more pairs of tiltable jaws. The controller may be configured to control the orientation (e.g., tilt angle) and position of each tiltable jaw relative to the radiation beam path. Through manipulating the orientation and position of each jaw, the radiation field may be tuned according to the shape, size and/or distance from a patient's skin of a patient target region. Additionally, a particular portion of a tumor may be targeted while limiting irradiation of healthy tissue (i.e., any non-target region). For example, if a tumor has a portion that is heterogeneous in geometry (e.g., the tumor has finger-like portions or projections extending in various directions), a pair of tiltable jaws may be oriented and positioned such that a portion of the radiation field has a reduced intensity or fluence level (e.g., feathered) in the region of the heterogeneous portion. Adjusting the tilt of the jaws may help reduce the amount of radiation delivered to the healthy tissue surrounding the heterogeneous tumor portion. If the patient target region has a portion that is homogenous and/or a prescribed dose over a patient target region is homogenous, the pair of tiltable jaws may be oriented and positioned such that the radiation field is conformal with an aperture defined between the pair of jaws in the region of the homogenous portion. For example, the pair of tiltable jaws may be adjusted such that the radiation field directed toward the homogenous portion of a patient target region may be “focused”, having a concentrated central field and sharp edges (e.g., smaller penumbra with a steeper fluence drop-off). Adjusting the tiltable jaws to sharply focus the radiation field may help deliver a higher and/or more concentrated dose of radiation to the homogenous tumor portion compared to an “unfocused” radiation field that has a penumbra portion with a more gradual fluence gradient (e.g., larger penumbra with a more gradual fluence drop-off).

Additionally, in some implementations, the one or more pairs of tiltable jaws can be configured to cull away scattered, low-energy photons from the edge of a radiation beam to prevent the low-energy photons from reaching and/or entering the skin of a patient. Low-energy photons may easily scatter in shallow regions just under a patient's skin (e.g., Compton's scatter) sooner after entering the skin, which may result in unintended irradiation of healthy tissue near the skin and hinder conformal radiation delivery. The one or more pairs of tiltable jaws may be adjusted and/or tuned to focus the beam and reduce the number of scattered, low-energy photons. This may help focus high-energy photons to the target region and/or better control the depth of the delivery dose by reducing photon scatter (e.g., Compton's scatter) in shallow regions just under a patient's skin. In some implementations, the one or more pairs of tiltable jaws may be used for shaping beams other than photon beams.

In some implementations, the one or more pairs of tiltable jaws can be positioned and/or oriented such that a radiation beam can be shaped based on a topography of a tumor or target, a context, and a proximity to a patient surface per firing angle of a radiation source, to help facilitate motion-tracking of a patient target region, focus the radiation fluence to central regions of a patient target region (e.g., voxels that are not near the target region boundary), sharpen the radiation field penumbra directed to the boundary regions of a patient target region (e.g., increased fluence gradient), and/or broaden the radiation field penumbra (e.g., reduced fluence gradient) for regions of a target region with heterogeneous tumor topography. Alternatively or additionally, tuning the position and/or orientation of one or more pairs of tiltable jaws may adjust the characteristics of the penumbra to help smooth compositing. For example, tuning the position and/or orientation of one or more pairs of tiltable jaws may help to mitigate the effects of dose rippling, threading, interplay, and/or any other patient and/or system motion artifacts that may affect a radiation dose. In some implementations, the one or more pairs of tiltable jaws can be positioned and/or oriented such that a radiation beam can be shaped to help compensate for radiation therapy system component limitations and/or faults. For example, the one or more pairs of tiltable jaws may be adjusted to help mitigate the effects on the therapeutic radiation field caused by pitting wear of electron target source material, movement of source spot on source target during treatment, migration of source spot on source target over life of the target, variations in the therapeutic radiation beam due to changes of a linac/gun, differences in the projected beam between different machines, non-homogenous fluence (e.g., with no flattening filter), machine (e.g., patient platform) deflection, and/or gantry bearing wear or damage. In some variations, the one or more pairs of tiltable jaws can be positioned and/or oriented such that a radiation beam can be shaped to improve treatment plan conformity and/or tumor motion conformity.

In some variations, to irradiate a central portion of a target region (e.g., a portion of a tumor spaced from an outer edge of the tumor), a pair of tiltable jaws may be moved laterally relative to one another under the control of a controller to alter the size of the aperture between the pair of tiltable jaws. For example, the pair of tiltable jaws may be moved toward one another to reduce the size of the aperture and thus reduce the size of a primary portion of the radiation field to be sufficiently small so as to irradiate the central portion of the target region without irradiating the edges of the target region. To create a sharp boundary between the portion of the target region being irradiated and the portion not being irradiated, the pair of tiltable jaws may be tilted under the control of a controller to change the shape of the aperture and reduce the size of the penumbra such that the penumbra does not extend significantly beyond the primary portion (e.g., such that no substantial penumbra exists in the radiation field). Radiation may then be delivered to the central portion of the target region through the aperture, avoiding the edge of the target region. Furthermore, if the target region has an edge that is clearly defined, the pair of tiltable jaws may be tilted and translated under the control of the controller to define an aperture having a shape and size such that a radiation beam passing through the aperture results in a radiation field having a sharp edge boundary aligning with the edge of the target region. Thus, the target region may be irradiated by the radiation field and the healthy tissue adjacent the target region may be avoided by the radiation beam.

In some variations, to irradiate a target region with heterogeneous edges, the pair of tiltable jaws may be manipulated by the controller to produce a radiation field having a primary portion and a penumbra portion extending away from an edge of the primary portion. The primary portion has a higher fluence than the penumbra portion. The radiation field may be aligned with the target region such that the heterogeneous edges are irradiated by the penumbra portion of the radiation field and a central portion of the target region is irradiated by the primary portion of the radiation field. Irradiating the heterogeneous edges with the penumbra portion of the radiation field may help reduce the exposure of healthy tissue adjacent the heterogeneous edges to radiation (e.g., compared to the exposure of healthy tissue if the heterogeneous edges are irradiated by the primary portion of the radiation field), while still irradiating the portion of the target region adjacent the healthy tissue. For example, to increase the size of the penumbra (e.g., the distance the penumbra extends from the edge of the primary region), the controller can cause the tiltable jaws to tilt relative to a central axis of an aperture of a primary collimator from which the radiation beam projects and/or relative to a central axis of a radiation beam extending from a radiation source to a tilt angle that causes the radiation field resulting from a radiation beam passing through the aperture to include a larger penumbra relative to the primary portion. Optionally and simultaneously or sequentially with the tilting, the pair of tiltable jaws may be laterally translated toward one another by the controller to decrease the size of the aperture and, thus, the size of the primary region of the radiation field and the size of the penumbra (e.g., an edge-to-edge distance of the penumbra).

In some variations, to achieve a larger penumbra portion while maintaining the size of the primary portion of a radiation field, the tilt angle of the pair of tiltable jaws may be increased under the control of the controller to increase the size of the penumbra portion. Simultaneously or sequentially, the pair of tiltable jaws may be translated toward each other to reduce the size of the aperture between them to maintain the size of the primary portion.

In some variations, to transition from irradiating a central homogenous portion of a target region to irradiating a portion of the target region having a heterogeneous edge, the pair of tiltable jaws may be laterally translated toward each other under the control of the controller to reduce the aperture size and, thus, the size of the primary portion of the radiation field. Simultaneously or sequentially, the tilt angle of one or both of the tiltable jaws may then be adjusted under the control of the controller to increase the size of the penumbra portion of the radiation field in the area of the heterogeneous edge.

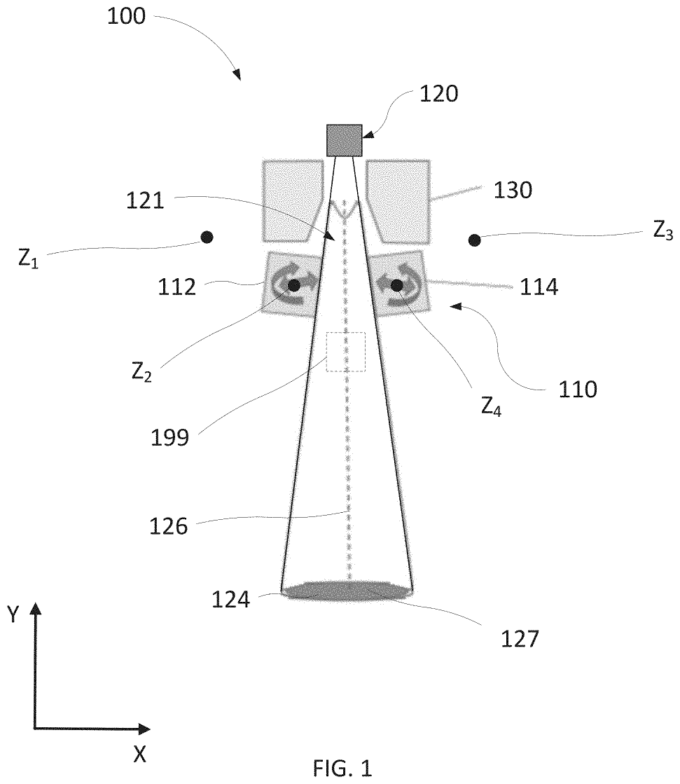

FIG. 1 is a schematic illustration of a radiation beam-shaping assembly 100. The radiation beam-shaping assembly 100 is configured to manipulate a radiation beam path to adjust characteristics of a radiation field 124. For example, the radiation field 124 may include a homogenous primary portion 127. The radiation beam-shaping assembly 100 may be configured to adjust the size of the primary portion. Furthermore, although not shown in FIG. 1, the radiation beam-shaping assembly 100 may be configured to adjust the radiation field 124 such that the radiation field 124 includes a penumbra portion extending from an outer edge of the primary portion 127. The radiation beam-shaping assembly 100 may be configured to adjust the size and shape of the penumbra portion relative to the primary portion 127. Furthermore, the radiation beam-shaping assembly 100 may be configured to shape a radiation beam such that the radiation field 124 has a sharp and clearly defined radiation field boundary (e.g., with a relatively small penumbra region or fast fluence gradient or drop-off from the primary portion 127 of the radiation field 124) or such that the radiation field 124 has a wider penumbra region (e.g., with a relatively larger penumbra region or a more gradual fluence gradient or drop-off from the primary portion 127 of the radiation field 124).

The radiation beam-shaping assembly 100 includes a pair of tiltable jaws 110. The pair of tiltable jaws 110 includes a first jaw 112 and a second jaw 114. Each of the first jaw 112 and the second jaw 114 is configured to be laterally movable relative to a radiation beam path under the control of a controller (not shown). For example, as shown in FIG. 1, the first jaw 112 and the second jaw 114 may be disposed in relation to a source of radiation 120 and a primary collimator 130 such that the first jaw 112 and the second jaw 114 may be moved (e.g., tilted and/or translated) relative to a path of a radiation beam 121 transmitted by or projecting from the source of radiation 120. For example, the first jaw 112 and the second jaw 114 may be disposed a distance from the source of radiation 120 between the source of radiation 120 and a patient target region (e.g., between the primary collimator 130 and the patient target region) and tiltable and/or translatable relative to a central axis 126 extending between the source of radiation 120 and the patient target region. The first jaw 112 and the second jaw 114 may be disposed opposite of each other about the central axis 126. The central axis 126 may be the central axis of a portion of the radiation beam 121 projecting from the primary collimator 130 prior to the radiation beam 121 reaching the pair of tiltable jaws 110. In some implementations, the central axis 126 may be the central axis of an aperture of the primary collimator 130 through which the radiation beam 121 is transmitted, and the first jaw 112 and the second jaw 114 may be disposed a distance along the central axis from the primary collimator 130 and/or the source of radiation 120.

In some implementations, the primary collimator 130 may be disposed relative to the source of radiation 120 such that the primary collimator 130 defines the path of the radiation beam 121 toward a target region (e.g., of a patient) to apply the radiation field 124 to the target region. For example, the primary collimator 130 may be disposed a distance from the source of radiation 120 along the central axis 126 such that the primary collimator 130 is disposed between the source of radiation 120 and the pair of tiltable jaws 110. The source of radiation 120 may be any type of ionizing radiation, for example, photon radiation (e.g., X-rays and gamma rays) and/or particle radiation (e.g., electrons, protons, neutrons, carbon ions, alpha particles, and beta particles). The first jaw 112 and the second jaw 114 may be arranged relative to the source of radiation 120 and the primary collimator 130 such that the first jaw 112 and the second jaw 114 may be moved relative to the path of the radiation beam 121 (e.g., relative to the central axis 126 of the radiation beam 121) to adjust the radiation field 124 of the target region (e.g., adjust the size of a primary region of the radiation field 124 and/or a penumbra of the radiation field 124). The first jaw 112 and the second jaw 114 may each include at least one collimation element. Each collimation element may be configured to obstruct at least a portion of a radiation beam when the collimation element is disposed in a path of the radiation beam. For example, each collimation element may be formed as a dense, solid body including attenuating materials (e.g., tungsten, tungsten alloy, lead, lead alloy, iron, iron alloy, steel alloy, or any other suitable alloy and/or high-impedance materials) configured to obstruct a radiation beam. Thus, the first jaw 112 and the second jaw 114 may be moved (e.g., tilted, rotated, or laterally shifted) toward and/or away from the central axis 126 of the radiation beam 121 to adjust the size and/or shape of an aperture through which the radiation beam 121 passes, thus adjusting the size and/or shape of the radiation field 124 by obstructing a portion of the radiation beam 121 from passing through each collimation element of the first pair of tiltable jaws 110. In some variations, a first portion of one or more of the jaws may be formed of a first attenuating material and a second portion of one or more of the jaws may be formed of a second lower attenuating material. For example, the second lower attenuating material may be disposed above the first attenuating material relative to the patient and closer to the source of radiation 120. When the one or more jaws is tilted into the beam path, the second lower attenuating material may be “toed into” the beam path upstream of the higher attenuating material to cull away lower energy photons (which may help reduce radiation dose deposition at regions near the skin and/or may reduce the fluence gradient across the penumbra region of the radiation field 124). The face of the first portion that faces the central axis 126 may be angled relative to the face of the second portion that faces the central axis 126 so that at certain tilt angles of the one or more jaws, the first portion does not interfere with the radiation beam path and the edge of the face of the second portion defines the aperture boundary. In some variations, the second lower attenuating material may include a cap formed of, for example, steel. In some implementations, each of the first jaw 112 and the second jaw 114 may include a cap or upper portion formed of a lower attenuating material than a base or lower portion of each of the first jaw 112 and the second jaw 114. The first jaw 112 and the second jaw 114 may be moved close enough to each other for the respective caps or upper portions to touch, nearly touch, or overlap to adjust the depth of the radiation beam across the entire field. Each collimation element may have any suitable shape and/or size. For example, each collimation element may include a flat central axis-facing surface. The angle formed between a central axis-facing surface and an upper surface of each collimation element (e.g., the surface that obstructs a portion of the radiation beam) may be, for example, about 90°. In some implementations, each collimation element may include one or more curved or faceted surfaces (e.g., a curved or multi-faceted central axis-facing surface).

In some implementations, the first jaw 112 may be configured to be rotatable about a first axis Z₁ (shown extending into/out of the plane of the page) such that an angle of a central axis-facing surface of the first jaw 112 relative to the central axis 126 may be adjusted (e.g., increased or decreased) and such that the first jaw 112 may move laterally towards and away from the central axis 126. The central axis 126 may be disposed in the X-Y plane and extend parallel to the Y-axis. The first axis Z₁ may extend perpendicularly to the X-Y plane. The first jaw 112 may be configured to also be rotatable (e.g., tiltable) about a second axis Z₂ (shown extending into the page) disposed perpendicular to the X-Y plane and parallel to the first axis Z₁. The angle of the radiation-facing surface of the first jaw 112 relative to the central axis 126 may be adjusted (e.g., increased or decreased) via tilting of the first jaw 112 about the second axis Z₂. For example, the angle of the radiation-facing surface of the first jaw 112 relative to the central axis 126 may be adjusted, for example, between about +/−5°, between about +/−2°, between about +/−3.11°, between about +/−0.93°, between about +/−0.62°, or within any other suitable range of angles. In some implementations, the second axis Z₂ may extend through the first jaw 112 and the second axis Z₂ may be configured to rotate about the first axis Z₁ based, at least in part, on the rotation of the first jaw 112 about the first axis Z₁. In some implementations, the first jaw 112 may be rotated about the first axis Z₁ and the second axis Z₂ simultaneously, i.e., as the second axis Z₂ rotates about the first axis Z₁. The first jaw 112 may be configured to translate laterally relative to the central axis 126 via a combined movement (e.g., a combined rotation) including a rotational movement about the first axis Z₁ and a counterrotational movement about the second axis Z₂ as the second axis Z₂ rotates about the first axis Z₁. The lateral movement of the first jaw 112 relative to the central axis 126 may be linear and/or curvilinear (e.g., arcuate). In some variations, the lateral movement of the first jaw 112 includes a motion component in a direction perpendicular to the central axis 126.

In some implementations, the second jaw 114 may be configured to be rotatable about a third axis Z₃ (shown extending into the page) such that an angle of a radiation-facing surface of the second jaw 114 relative to the central axis 126 may be adjusted (e.g., increased or decreased) and such that the second jaw 114 may move laterally towards and away from the central axis 126. The third axis Z₃ may extend perpendicular to the X-Y plane. The second jaw 112 may be configured to also be rotatable (e.g., tiltable) about a fourth axis Z₄ (shown extending into the page) disposed perpendicular to the X-Y plane and parallel to the third axis Z₃. The angle of the radiation-facing surface of the second jaw 114 relative to the central axis 126 may be adjusted (e.g., increased or decreased) via tilting of the second jaw 114 about the fourth axis Z₄. For example, the angle of the radiation-facing surface of the second jaw 114 relative to the central axis 126 may be adjusted, for example, between about +/−5°, between about +/−2°, between about +1-3.11°, between about +/−0.93°, between about +/−0.62°, or within any other suitable range of angles. In some implementations, the third axis Z₃ and the fourth axis Z₄ may be disposed parallel to the first axis Z₁ and the second axis Z₂. In some implementations, the fourth axis Z₄ may be configured to rotate about the third axis Z₃ based, at least in part, on the rotation of the second jaw 114 about the third axis Z₃. In some implementations, the second jaw 114 may be rotated about the third axis Z₃ and the fourth axis Z₄ simultaneously, i.e., as the fourth axis Z₄ rotates about the third axis Z₃. The second jaw 114 may be configured to translate laterally relative to the central axis 126 via a combined movement including a rotational movement about the third axis Z₃ and a rotational movement about the fourth axis Z₄ as the fourth axis Z₄ rotates about the third axis Z₃. The lateral movement of the second jaw 114 relative to the central axis 126 may be linear and/or curvilinear (e.g., arcuate). In some variations, the lateral movement of the second jaw 114 includes a motion component in a direction perpendicular to the central axis 126.

In some embodiments, rather than being rotatable about the first axis Z₁ and the second axis Z₂, the first jaw 112 may be configured to be rotatable about the first axis Z₁ and to be laterally translated along a first lateral path that intersects the central axis 126. The first lateral path may be disposed perpendicular to the central axis or at a non-perpendicular and non-zero angle relative to the central axis 126. In some implementations, at least a component of the lateral motion of the first jaw 112 may be perpendicular to the central axis 126. In some implementations, the first lateral path may be arcuate or curvilinear. In some implementations, the first lateral path may be linear. Additionally, rather than being rotatable about the third axis Z₃ and the fourth axis Z₄, the second jaw 114 may be configured to be rotatable about the third axis Z₃ and to be laterally translated along a second lateral path that intersects the central axis 126. The second lateral path may be disposed perpendicular to the central axis or at a non-perpendicular and non-zero angle relative to the central axis 126. In some implementations, the second lateral path may mirror the first lateral path about the central axis 126. In some implementations, at least a component of the lateral motion of the second jaw 114 may be perpendicular to the central axis 126. In some implementations, the second lateral path may be arcuate or curvilinear. In some implementations, the second lateral path may be linear.

In some implementations, the first jaw 112 and the second jaw 114 may be moved in concert such that the first jaw 112 and the second jaw 114 tilts and/or laterally translate simultaneously but in opposite directions. For example, as the first jaw 112 moves laterally toward the central axis 126, the second jaw 114 may move laterally toward the central axis 126 by the same distance and in the opposite direction of the first jaw 112. As the first jaw 112 moves laterally away from the central axis 126, the second jaw 114 may laterally translate away from the central axis 126 by the same distance and in the opposite direction of the first jaw 112. As the first jaw 112 tilts about the first axis Z₁ and/or the second axis Z₂, the second jaw 114 may tilt about the third axis Z₃ and/or the fourth axis Z₄, respectively, in the opposite rotational direction as the first jaw 112 tilts. In some implementations, the first jaw 112 and the second jaw 114 may be laterally and/or rotationally moved independently of each other.

In some embodiments, the radiation beam-shaping assembly 100 may include a group of actuators (not shown). Any suitable number and type of actuators may be configured to control the rotation and/or lateral translation of the first jaw 112 and the second jaw 114. Each of the actuators from the group of actuators may be, for example, rotary actuators. In some implementations, the group of actuators may include linear actuators. Each of the actuators from the group of actuators may include and/or be coupled to mechanical, electro-mechanical, and/or software systems. For example, each of the actuators from the group of actuators may be operatively coupled to the controller such that the actuators control the rotation and/or lateral translation of the first jaw 112 and the second jaw 114 under the control of the controller. A first actuator from the group of actuators may be coupled to the first jaw 112 and may be configured to rotate the first jaw 112 about the first axis Z₁. A second actuator from the group of actuators may be coupled to the first jaw 112 and configured to rotate the first jaw 112 about the second axis Z₂ and/or translate the first jaw 112 laterally relative to the central axis 126. A third actuator from the group of actuators may be coupled to the second jaw 114 and may be configured to rotate the second jaw 114 about the third axis Z₃. A fourth actuator from the group of actuators may be coupled to the second jaw 114 and configured to rotate the second jaw 114 about the fourth axis Z₄ and/or translate the second jaw 114 laterally relative to the central axis 126. In some implementations, the group of actuators may include a first linear actuator coupled to the first jaw 112 and configured to translate the first jaw 112 laterally relative to the central axis 126. A first rotary actuator may be coupled to (e.g., mounted to) the first linear actuator and configured to rotate the first jaw 112 to change the tilt of the first jaw 112 relative to the central axis 126. A second linear actuator can be coupled to the second jaw 114 and configured to translate the second jaw 114 laterally relative to the central axis 126. A second rotary actuator may be coupled to (e.g., mounted to) the second linear actuator and configured to rotate the second jaw 114 to change the tilt of the second jaw 114 relative to the central axis 126.

For example, in some implementations, the group of actuators may include an actuator for each rotational axis about which each of the first jaw 112 and the second jaw 114 may be rotated. For example, in some implementations, the group of actuators may include a first actuator configured to rotate the first jaw 112 about the first axis Z₁ and a second actuator configured to rotate the first jaw 112 about the second axis Z₂. The group of actuators may include a third actuator configured to rotate the second jaw 114 about the third axis and a fourth actuator configured to rotate the second jaw 114 about the fourth axis Z₄.

In some implementations, the group of actuators may include a first actuator configured to rotate the first jaw 112 about the first axis Z₁ and a second actuator configured to translate the first jaw 112 laterally relative to the central axis 126. The group of actuators may include a third actuator configured to rotate the second jaw 114 about the third axis and a fourth actuator configured to translate the second jaw 114 laterally relative to the central axis 126.

In some implementations, the first jaw 112 may be included in a first carriage (not shown). The first jaw 112 may be configured to be rotated or tilted relative to the first carriage and/or with the first carriage. For example, in some implementations, the first jaw 112 may be rotated about the first axis Z₁ relative to the first carriage and may be coupled to the first carriage such that rotation of the first carriage about the second axis Z₂ causes rotation of the first jaw 112 about the second axis Z₂. Lateral translation of the first jaw 112 relative to the central axis 126 may be accomplished via simultaneous rotation of the first jaw 112 about the first axis Z₁ and rotation of the first carriage about the second axis Z₂ such that the first jaw 112 (and the first axis Z₁) also rotates about the second axis Z₂. In some implementations, the first carriage and the first jaw 112 may translate laterally relative to the central axis 126 (e.g., the distance between the first jaw 112 and the central axis 126 may increase or decrease) and the orientation of the first carriage and the first jaw 112 may remain the same relative to the central axis 126 due to the simultaneous rotation of the first jaw 112 about the first axis Z₁ and the second axis Z₂.

In some implementations, the second jaw 114 may be included in a second carriage (not shown). The second jaw 114 may be configured to be rotated or tilted relative to the second carriage and/or with the second carriage. For example, in some implementations, the second jaw 114 may be rotated about the third axis Z₃ relative to the second carriage and may be coupled to the second carriage such that rotation of the second carriage about the third axis Z₃ caused rotation of the second jaw 114 about the fourth axis Z₄. Lateral translation of the second jaw 114 relative to the central axis 126 may be accomplished via simultaneous rotation of the second jaw 114 about the third axis Z₃ and rotation of the second carriage about the fourth axis Z₄ such that the second jaw 114 (and the third axis Z₃) also rotates about the fourth axis Z₄, resulting in the second carriage and the second jaw 114 translating laterally relative to the central axis 126. In some implementations, the second carriage and the second jaw 114 may translate laterally relative to the central axis 126 (e.g., the distance between the second jaw 114 and the central axis 126 may increase or decrease) and the orientation of the second carriage and the second jaw 114 may remain the same relative to the central axis 126 due to the simultaneous rotation of the second jaw 114 about the third axis Z₃ and the fourth axis Z₄.

In some implementations, the radiation beam-shaping assembly 100 may be configured to be mounted on a rotatable gantry of a radiation therapy system. In some implementations, as shown in FIG. 1, a multi-leaf collimator assembly 199 may optionally be disposed in the path of the radiation beam 121. Although shown in FIG. 1 as being located below the first jaw 112 and the second jaw 114 relative to the source of radiation 120, in some implementations the multi-leaf collimator assembly 199 may be located above, below, or between the first jaw 112 and the second jaw 114.

In use, adjustments to the orientation and/or position of each of the first jaw 112 and the second jaw 114 may adjust the focus and/or other characteristics of the radiation field 124. For example, the first jaw 112 and the second jaw 114 may be rotated and/or laterally translated to adjust characteristics of the radiation field 124 such as a shape and/or size of the primary radiation field, penumbra shape, penumbra size, penumbra fluence, energy delivery depth and/or the softness or hardness of a knee (e.g., the transition region between the primary and penumbra regions). For example, tilting the first jaw 112 may adjust the shape and sharpness of the penumbra of the radiation beam 121 on or below the patient surface. For example, increasing the tilt angle of a radiation-facing surface of each of the first jaw 112 and the second jaw 114 relative to the central axis 126 may cause a penumbra of a radiation field to increase in size and/or the edges of the penumbra or the primary region to be more gradual, while reducing the tilt angle of the radiation-facing surface of each of the first jaw 112 and the second jaw 114 relative to the central axis 126 may cause a penumbra of a radiation field to decrease in size and/or the edges of the penumbra or the primary region to sharpen. In some implementations, the radiation field 124 and associated characteristics may be symmetrical. In some implementations, the radiation field 124 and associated characteristics may be asymmetrical to treat target regions (e.g., tumors) having asymmetrical shapes and/or asymmetrical portions of target regions (e.g., portions of tumors).

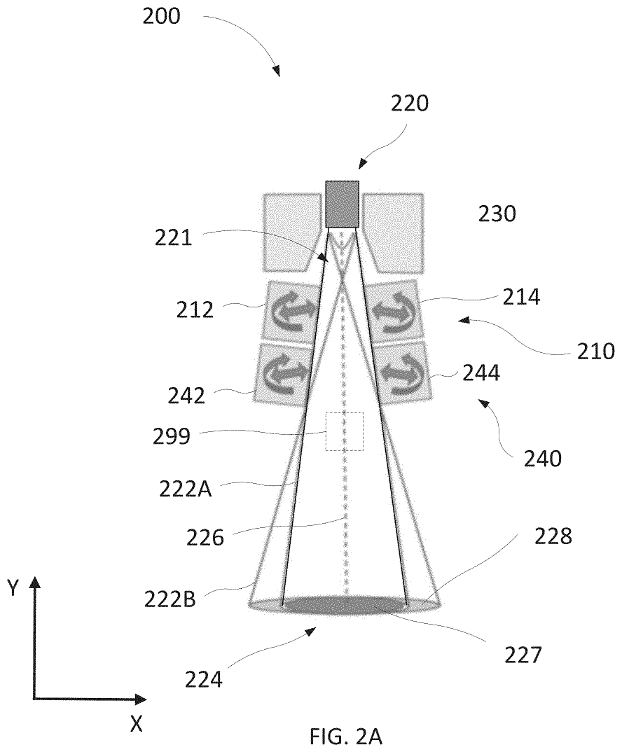

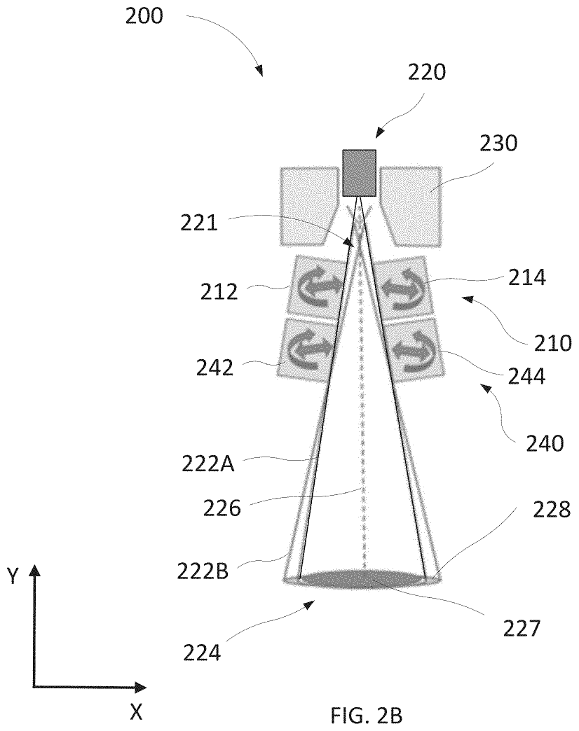

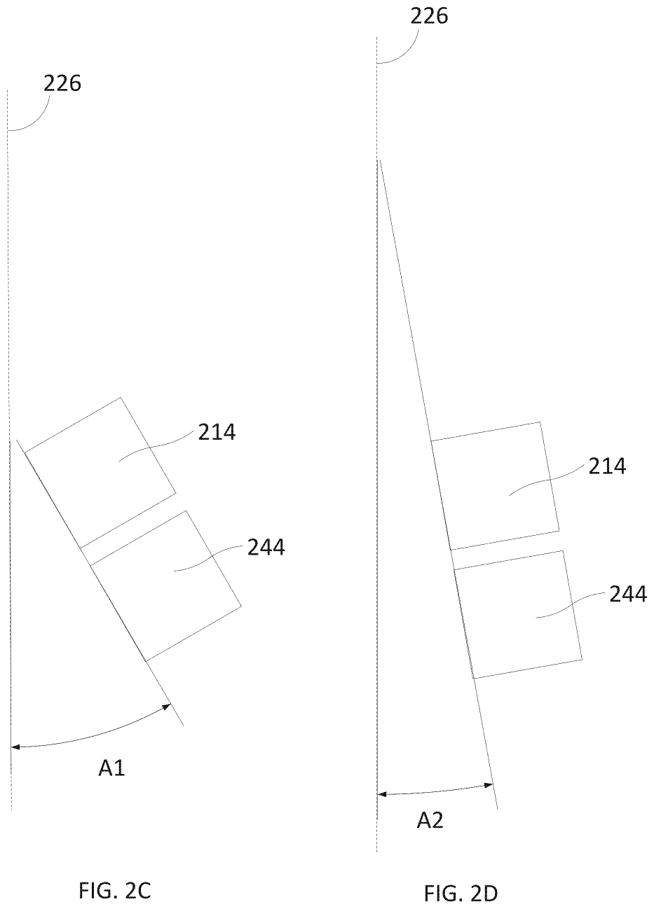

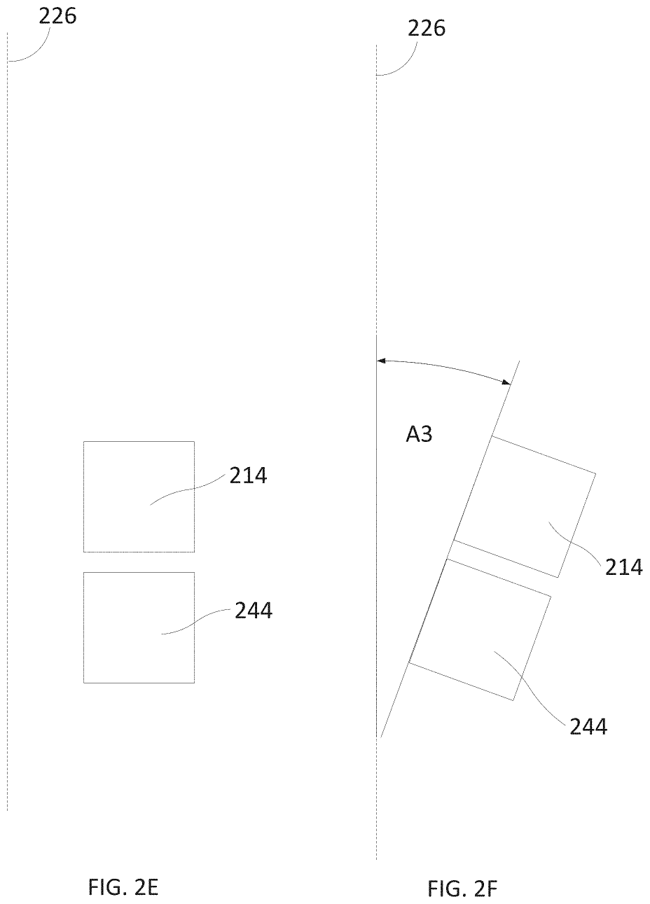

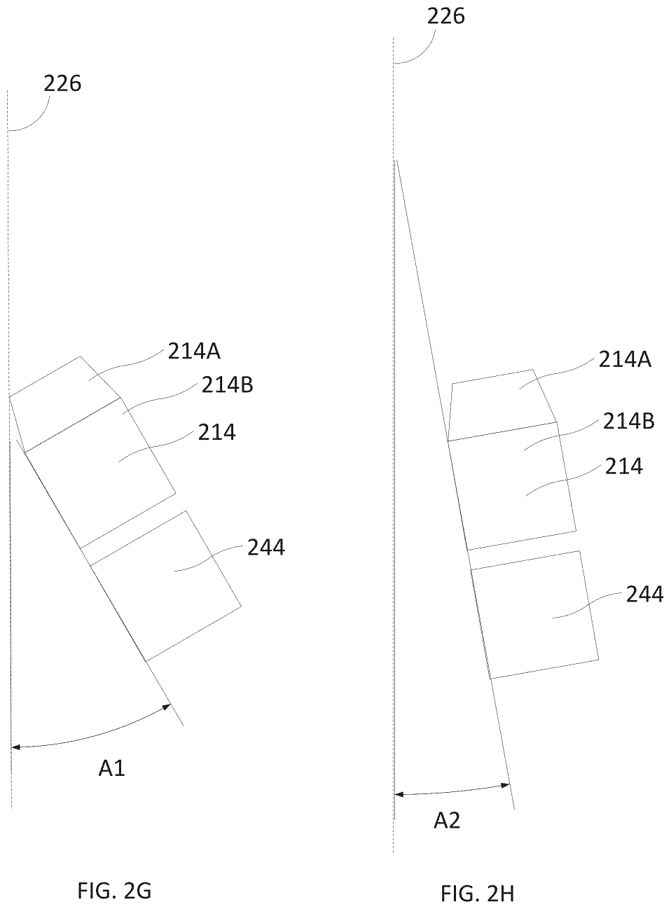

Although FIG. 1 shows only one pair of tiltable jaws, the radiation beam-shaping assemblies described herein may include any suitable number of pairs of tiltable jaws. For example, FIGS. 2A and 2B are schematic illustrations of a radiation beam-shaping assembly 200 in a first configuration and a second configuration, respectively. The radiation beam-shaping assembly 200 may be the same or similar in structure and/or function to any of the radiation beam-shaping assemblies described herein, such as, for example, the radiation beam-shaping assembly 100 described above with respect to FIG. 1. For example, the radiation beam-shaping assembly 200 includes a first pair of tiltable jaws 210 that may be the same or similar in structure and/or function to the tiltable jaws 110. The tiltable jaws 210 include a first jaw 212 and a second jaw 214. Each of the first jaw 212 and the second jaw 214 is laterally movable relative to a radiation beam path. For example, as shown in FIG. 2A, the first jaw 212 and the second jaw 214 may be disposed in relation to a source of radiation 220 and a primary collimator 230 such that the first jaw 212 and the second jaw 214 may be moved (e.g., tilted and/or translated) under the control of a controller (not shown) relative to a path of a radiation beam 221 projecting from the source of radiation 220. The first jaw 212 and the second jaw 214 may be disposed opposite of each other about a central axis 226. The central axis 226 may be the central axis of the portion of the radiation beam 221 projecting from the primary collimator 230 prior to the radiation beam 221 reaching the first pair of tiltable jaws 210. In some implementations, the central axis 226 may be the central axis of an aperture of the primary collimator 230 through which the radiation beam 221 is transmitted, and the first jaw 212 and the second jaw 214 may be disposed a distance along the central axis from the primary collimator 230 and/or the source of radiation 220.

The radiation beam-shaping assembly 200 also includes a second pair of tiltable jaws 240. The second pair of tiltable jaws 240 may be the same or similar in structure and/or function to the first pair of tiltable jaws 210 and to any of the pairs of tiltable jaws described herein. For example, the second pair of tiltable jaws 240 includes a first jaw 242 and a second jaw 244. Each of the first jaw 242 and the second jaw 244 is laterally translatable and rotationally movable relative to the central axis 226 of the radiation beam 221.

The first pair of tiltable jaws 210 (e.g., the first jaw 212 and the second jaw 214) may be an upper pair of tiltable jaws and the second pair of tiltable jaws 240 (e.g., the first jaw 242 and the second jaw 244) may be a lower pair of tiltable jaws. Additionally, the first pair of tiltable jaws 210 and the second pair of tiltable jaws 240 may each include a jaw on a first side of the central axis 226 (e.g., the left side as shown in FIG. 2A) and a jaw on a second side of the central axis 226 (e.g., the right side as shown in FIG. 2A) opposite the first side. Thus, the first jaw 212 of the first upper pair of tiltable jaws 210 and the first jaw 242 of the second lower pair of tiltable jaws 240 may be disposed on the left side of the central axis 226 and may be referred to as a left upper jaw 212 and a left lower jaw 242, respectively. The second jaw 214 of the first upper pair of tiltable jaws 210 and the second jaw 244 of the second lower pair of tiltable jaws 240 may be disposed on the right side of the central axis 226 and may be referred to as a right upper jaw 214 and a right lower jaw 244, respectively.

Each jaw of the first pair of tiltable jaws 210 and each jaw of the second pair of tiltable jaws 240 include at least one collimation element. Each collimation element may be configured to obstruct at least a portion of the radiation beam 221 when the collimation element is disposed in a path of the radiation beam 221. For example, each collimation element may be formed as a dense, solid body (e.g., formed of tungsten) configured to obstruct at least a portion of the radiation beam 221. Thus, the left upper jaw 212, the right upper jaw 214, the left lower jaw 242, and the right lower jaw 244 may be moved (e.g., tilted, rotated, or laterally shifted) toward and/or away from the central axis 226 of the radiation beam 221 to adjust the size and/or shape of an aperture through which the radiation beam 221 passes, thus adjusting the size, shape, and/or other characteristics (e.g., fluence and/or energy delivery depth) of the radiation field 224 by obstructing a portion of the radiation beam 221 from passing through each collimation element of the first pair of tiltable jaws 210 and the second pair of tiltable jaws 240. Each collimation element may have any suitable shape and/or size. For example, each collimation element may include one or more flat or curved surfaces. In some implementations, the system 200 may include a first pair of jaws having curved surfaces such that the first pair of jaws can create a uniform penumbra irrespective of a size of the radiation field 224 in a first mode. The system 200 may have a second mode in which the first pair of jaws having curved surfaces can be retracted relative to the central axis of the beam such that a second pair of jaws having flat surfaces to adjust the fluence, resolving power, or amount of low energy photons of the radiation field 224.

For example, as shown in FIG. 2A, the first pair of tiltable jaws 210 and the second pair of tiltable jaws 240 are arranged relative to the primary collimator 230 and the source of radiation 220 such that the first pair of tiltable jaws 210 and the second pair of tiltable jaws 240 shape the radiation beam 221 to a first shape and size 222A as the radiation beam 221 passes through the aperture defined by the first pair of tiltable jaws 210 and the second pair of tiltable jaws 240. As a result of the first pair of tiltable jaws 210 and the second pair of tiltable jaws 240 obstructing the radiation beam 221 such that a first portion 222A of the radiation beam 221 has a first shape and size, the radiation field 224 has a primary region 227 that has a substantially constant fluence. The radiation field 224 may have a secondary region 228 (e.g., a penumbra portion) surrounding and/or extending away from the primary region 227 formed by a second portion 222B of the radiation beam 221 that is unobstructed geometrically, scatters outside of the first portion 222A, and/or includes beam energy transmitted through a portion of a jaw of the first pair of tiltable jaws 210 and/or the second pair of tiltable jaws 240 (e.g., transmission penumbra). The secondary region 228 may have a reduced fluence and at a different energy delivery depth potential than the primary region 227. The secondary region 228 may be concentric with the primary region 227 and symmetrically shaped or may extend away from an outer perimeter of the primary region 227 asymmetrically. As shown in FIG. 2A, the radiation beam 221 may include rays that project outside of the primary region 227 forming the secondary region 228.

The first pair of tiltable jaws 210 and the second pair of tiltable jaws 240 may be rotated and/or translated relative to the radiation beam 221 to adjust the radiation field 224 (e.g., tune the shape and size of the primary region 227 and the secondary region 228). As shown in FIG. 2B, the left upper jaw 212 and the right upper jaw 214 of the first pair of tiltable jaws 210 may be translated relative to the central axis 226 of the radiation beam 221 and closer to each other, such that an aperture defined between the left upper jaw 212 and the right upper jaw 214 is smaller compared to when the left upper jaw 212 and the right upper jaw 214 are farther apart from each other. As the aperture decreases in size, the portion of the radiation beam 221 able to travel past the first pair of tiltable jaws 210 decreases because more of the radiation beam 221 is obstructed by the first pair of tiltable jaws 210. Additionally, each jaw of the first pair of tiltable jaws 210 may be tilted toward the central axis 226 of the radiation beam 221 such that an upper edge of each jaw is closer to the central axis 226 and the opposite jaw, therefore further reducing the size of the aperture and changing the shape defined between the first pair of tiltable jaws 210. Furthermore, the left lower jaw 242 and the right lower jaw 244 of the second pair of tiltable jaws 240 may be translated relative to the central axis 226 of the radiation beam 221 and closer to each other, such that an aperture defined between the left lower jaw 242 and the right lower jaw 244 is smaller compared to when the left lower jaw 242 and the right lower jaw 244 are farther apart from each other, allowing a reduced portion of the radiation beam 241 to pass between the second pair of tiltable jaws 240. Additionally, each jaw of the second pair of tiltable jaws 240 may be tilted toward the central axis 226 of the radiation beam 221 such that an upper edge of each jaw is closer to the central axis 226 and the opposite jaw, therefore further reducing the size of the aperture and changing the shape defined between the second pair of tiltable jaws 240.

As a result of the first pair of tiltable jaws 210 and the second pair of tiltable jaws 240 being transitioned to obstruct more of the radiation beam 221 and/or to obstruct the radiation beam 221 in a different orientation than in the first configuration shown in FIG. 2A, the first portion 222A of the radiation beam and/or the second portion 222B of the radiation beam may be transitioned to a second shape and size such that the radiation field 224 has different characteristics compared to the radiation field 224 shown in FIG. 2A. For example, the size, outer boundary, and other characteristics of the primary region 227 may be based, at least in part, on the location of a bottom edge of the left lower jaw 242 and a bottom edge of the right lower jaw 242 nearest the central axis 226 of the radiation beam 221. The size, outer boundary, sharpness, and other characteristics of the secondary region 228 (e.g., the penumbra) may be based, at least in part, on the orientation of the first pair of tiltable jaws 210 and the second pair of tiltable jaws 240 relative to the central axis 226 of the radiation beam 228. Thus, as shown in FIG. 2B, translating the first pair of tiltable jaws 210 closer to the central axis 226 and tilting the upper edges of the first pair of tiltable jaws 210 and the second pair of tiltable jaws 240 toward the central axis 226 may result in the primary region 227 remaining the same size as in the first configuration of the assembly 200 shown in FIG. 2A, but the size of the secondary region 228 may be decreased.

In some implementations, the left jaws of the lower pair of tiltable jaws 240 and the upper pair of tiltable jaws 210 may be configured to move in concert with each other. In some variations, all of the jaws disposed on a first side (e.g., the left side) of the central axis 226 of the radiation beam 228 may be configured to move in concert with each other, and all of the jaws on a second side (e.g., the right side) of the central axis 226 may be configured to move in concert with each other. For example, the left lower jaw 242 may be configured to move in concert with the left upper jaw 212 such that the jaws on the left side are moved (e.g., rotated and/or translated) together and by the same amount. Similarly, the right lower jaw 244 may be configured to move in concert with the right upper jaw 214 such that the jaws on the right side of the central axis 226 are moved (e.g., rotated and/or translated) together and by the same amount. In some implementations, the right upper jaw 212 and the right lower jaw 242 may be mounted on a first carriage and the left upper jaw 214 and the left lower jaw 244 may be mounted on a second carriage. Alternatively or additionally, the jaws disposed on the right side of the central axis 226 of the radiation beam 228 may be configured to move in concert with the jaws disposed on the left side of the central axis 226. In some variations, the jaws disposed on the right side of the central axis 226 of the radiation beam 228 may be configured to move independently of the jaws disposed on the left side of the central axis 226.

In some implementations, the left lower jaw 242 and the left upper jaw 212 are configured to move (e.g., rotate and/or translate) independently of each other, and the right lower jaw 244 and the right upper jaw 214 are configured to move (e.g., rotate and/or translate) independently of each other. In some implementations, each jaw of the first pair of tiltable jaws 210 and the second pair of tiltable jaws 240 may be mounted in an individual carriage for rotational and/or translational movement.

Although the radiation beam-shaping assembly 200 shows the first pair of tiltable jaws 210 and the second pair of tiltable jaws 240, the radiation beam-shaping assembly 200 may include any suitable number of tiltable jaws of pairs of tiltable jaws. Furthermore, although the radiation beam-shaping assembly 200 shows a first pair of tiltable jaws 210 and a second pair of tiltable jaws 240 positioned on opposite sides of a radiation beam, in some embodiments a radiation beam-shaping assembly may include a stationary jaw or collimation element positioned on a first side of a radiation beam and one or more tiltable and/or translatable jaws disposed on a second side of the radiation beam (e.g., above or below each other with respect to the source of radiation and the patient target region). In some variations, the first pair of tiltable jaws 210 may be tiltable and/or translatable and the second pair of tiltable jaws 240 may be fixed such that the second pair of tiltable jaws 240 defines an aperture having a constant shape and size.

In some variations, jaws that are disposed up-stream of other jaws relative to the source of radiation 220 may be formed of or include lesser attenuating materials compared to down-stream jaws. For example, collimation elements of the first jaw 212 and the second jaw 214 of the first pair of tiltable jaws 210 may be formed of a material that is less attenuating than collimation elements of the first jaw 242 and the second jaw 244 of the second pair of tiltable jaws 240. In variations including additional pairs of tiltable jaws, each pair of tiltable jaws may be formed of or include a higher attenuating material than an adjacent pair of tiltable jaws upstream of the pair of tiltable jaws.

The first pair of tiltable jaws 210 and the second pair of tiltable jaws 240 may be disposed any suitable distance from each other. For example, the distance between the first pair of tiltable jaws 210 and the second pair of tiltable jaws 240 along the central axis 226 may be a greater distance than is required for mechanical clearance of the first pair of tiltable jaws 210 relative to the second pair of tiltable jaws 240 when one or both are being adjusted (e.g., translated and/or rotated). In some variations, the distance between the first jaw 212 and the first jaw 242, for example, may be about fifty to about two hundred millimeters greater (e.g., one hundred fifty millimeters greater) than needed for each jaw to translate and/or rotate without contacting the other jaw. In some variations, the second pair of tiltable jaws 240 may be disposed, for example, between about 300 mm and about 800 mm from a downstream or exit side of the patient (i.e., the side of the patient opposite the source of radiation 220). In some variations, the first pair of tiltable jaws 210 and the second pair of tiltable jaws 240 may be disposed any distance apart along the central axis 226 such that a particular characteristic of a radiation field may be achieved. For example, the first pair of tiltable jaws 210 may be disposed a distance from the source of radiation 220 that is equal to between about 6% and about 24% of the distance from the source of radiation 220 to the surface of the patient or to the target region. The second pair of tiltable jaws 240 may be disposed a distance from the patient that is as small as possible but still allows for adequate patient clearance. For example, the distance of the second pair of tiltable jaws 240 may be sufficient to allow for patient set up and/or may be as close as possible to the patient without touching the patient during therapeutic radiation delivery. In some variations, the second pair of tiltable jaws 240 may contact the patient during therapeutic radiation delivery.

In some implementations, the first pair of tiltable jaws 210 may be disposed at a sufficient distance from the second pair of tiltable jaws 240 such that the second pair of tiltable jaws 240 may shape the radiation beam 221 to cause the radiation field 224 to have a sharper edge (e.g., of the primary region 227 and/or the secondary region 228) than would have resulted from shaping the radiation beam 221 with only the first pair of tiltable jaws 210. In some implementations, for example, the first pair of tiltable jaws 210 may be disposed between the second pair of tiltable jaws 240 and the source of radiation 220 such that the first pair of tiltable jaws 210 may be controlled to adjust the size of the secondary region 228 (e.g., the penumbra portion), and the second pair of tiltable jaws 240 may be disposed close to the patient (e.g., as close as possible to the patient while providing adequate clearance with the patient) such that the second pair of tiltable jaws 240 may be used to control the sharpness of an edge of the radiation field 224 (e.g., such that the edge of the radiation field 224 is sharper than if the beam is shaped by the first pair of tiltable jaws 210 alone).

In some implementations, the beam-facing surface of a jaw (e.g., one or both jaws) of the first pair of tiltable jaws 210 may be disposed offset from the beam-facing surface of the jaw of the second pair of tiltable jaws 240 disposed on the same side of the radiation beam 221 as the jaw of the first pair of tiltable jaws 210. For example, a beam-facing surface of a jaw of the first pair of tiltable jaws 210 (e.g., the right upper jaw 214) may be disposed farther from the central axis 226 than a beam-facing surface of a jaw of the second pair of tiltable jaws 240 on the same side of the radiation beam 221 as the jaw of the first pair of tiltable jaws 210 (e.g., the right lower jaw 244). The first pair of tiltable jaws 210 may be disposed at a sufficient distance from the second pair of tiltable jaws 240 and may have one or more beam-facing surfaces that are sufficiently offset relative to beam-facing surfaces of the second pair of tiltable jaws 240 on the same side of the radiation beam 221 such that the second pair of tiltable jaws 240 may shape the radiation beam 221 to cause the radiation field 224 to have a sharper edge (e.g., of the primary region 227 and/or the secondary region 228) than if the radiation field 224 had been shaped by the first pair of tiltable jaws 210 and the second pair of tiltable jaws 240 in a configuration in which the beam-facing surfaces of the jaws on one or both sides of the radiation beam 221 are coplanar. In some variations, the locations of the first pair of tiltable jaws 210 and the second pair of tiltable jaws 240 relative to the central axis 226 and/or each other may be adjusted via one or more actuators configured to translate the first pair of tiltable jaws 210 and/or the second pair of tiltable jaws 240 along the central axis 226 toward and/or away from the source of radiation 220.

In some implementations, the radiation beam-shaping assembly 200 may be configured to be mounted on a rotatable gantry of a radiation therapy system. In some implementations, as shown in FIG. 2A, a multi-leaf collimator assembly 299 may optionally be disposed in the path of the radiation beam 221. Although shown in FIG. 2A as being located below the first pair of tiltable jaws 210 and the second pair of tiltable jaws 240, in some implementations the multi-leaf collimator assembly 299 may be located above, below, or between the first pair of tiltable jaws 210 and the second pair of tiltable jaws 240. In some implementations, the multi-leaf collimator assembly 299 and the second pair of tiltable jaws 240 may be disposed any suitable distance relative to each other (e.g., both can be disposed as close to the patient as possible with enough space for any mechanical clearance needed for adjustment of the multi-leaf collimator assembly 299 and/or the second pair of tiltable jaws 240). In implementations in which a multi-leaf collimator assembly 299 is disposed between the first pair of tiltable jaws 210 and the second pair of tiltable jaws 240, the first pair of tiltable jaws 210 may be disposed a greater distance from the multi-leaf collimator assembly 299 than is required for mechanical clearance of the first pair of tiltable jaws 210 relative to the multi-leaf collimator assembly 299 when the first pair of tiltable jaws 210 is being adjusted (e.g., translated and/or rotated).

FIGS. 2C-2F are schematic illustrations of the right upper jaw 214 of the first pair of tiltable jaws 210 and the right lower jaw 244 of the second pair of tiltable jaws 240 in various exemplary tilt angle configurations relative to the central axis 226. In some implementations, the right upper jaw 214 and the right lower jaw 244 may be configured to tilt and/or rotate between the various configurations shown in FIGS. 2C-2F (e.g., under the control of the controller), pausing at any suitable orientation for irradiation of a patient target region. Each of the configurations of the right upper jaw 214 and the right lower jaw 244 shown in FIGS. 2C-2F may correspond to particular characteristics of the radiation field 224 (e.g., penumbra size and fluence gradient).

FIG. 2C shows the right upper jaw 214 and the right lower jaw 244 oriented at a first tilt angle A1 relative to the central axis 226 (e.g., oriented such that a beam-facing surface of each of the right upper jaw 214 and the right lower jaw 244 are oriented at the first tilt angle A1 relative to the central axis 226). FIG. 2D shows the right upper jaw 214 and the right lower jaw 244 oriented at a second tilt angle A2 relative to the central axis 226 (e.g., oriented such that the beam-facing surface of each of the right upper jaw 214 and the right lower jaw 244 are oriented at the second tilt angle A2 relative to the central axis 226). The second tilt angle A2 is smaller than the first tilt angle A1. FIG. 2E shows the right upper jaw 214 and the right lower jaw 244 oriented such that the beam-facing surfaces of the right upper jaw 214 and the right lower jaw 244 are parallel (i.e., oriented at a 0° angle) relative to the central axis 226. FIG. 2F shows the right upper jaw 214 and the right lower jaw 244 oriented at a third tilt angle A3 relative to the central axis 226. The third tilt angle A3 is oriented opposite of the first tilt angle A1 and the second tilt angle A2, and may be a negative angle relative to the first tilt angle A1 and the second tilt angle A2. In some implementations, the upstream edge of the right upper jaw 214 may be “toed into” the radiation beam to cull away lower energy photons and harden the edge of the penumbra of the radiation field 224. For example, the right upper jaw 214 can be tilted toward the central axis 226 as shown in FIGS. 2C and 2D and then translated toward the central axis 226.

Although FIGS. 2C-2F show only the right upper jaw 214 and the right lower jaw 244, the left upper jaw 212 of the first pair of tiltable jaws 210 and the left lower jaw 242 of the second pair of tiltable jaws 240 may be configured to tilt and/or rotate (e.g., under the control of the controller) between various configurations similar, but opposite to (e.g., mirror symmetric fashion), the tilt angle configurations shown in FIGS. 2C-2F relative to the central axis 226. For example, the left upper jaw 212 and the left lower jaw 242 may be configured to tilt and/or rotate to an orientation in which a beam-facing surface of each of the left upper jaw 212 and the left lower jaw 242 are disposed at a tilt angle relative to the central axis 226 of negative A1, negative A2, a 0° angle, and/or negative A3. Furthermore, in some variations, the left upper jaw 212, the left upper jaw 214, the left lower jaw 242, and the right lower jaw 244 may each be configured to tilt and/or rotate to different tilt angles relative to the central axis 226.

In some implementations, a first portion of each of the first pair of tiltable jaws 210 may be formed of a first attenuating material and a second portion of each of the first pair of tiltable jaws 210 may be formed of a second lower attenuating material. For example, as shown in FIGS. 2G and 2H, the right upper jaw 214 may include a first portion 214A and a second portion 214B. The first portion 214A may include a second lower attenuating material and the second portion 214B may include a first higher attenuating material. The first portion 214A may be disposed on an upper surface of the second portion 214B and closer to the source of radiation 120 than the second portion 214B. As shown in FIG. 2G, when the right upper jaw 214 tilted toward the central axis 226 and into the beam path, the first portion 214A can be “toed into” (i.e., tilted to increase the tilt angle A1 and translated toward the central axis 226) the beam path upstream of the higher attenuating material to cull away lower energy photons that would provide less dose nearer the skin and/or to cause the penumbra region of the radiation field 224 to have a more gradual fluence change. The face of the first portion 214A that faces the central axis 126 may be angled relative to the face of the second portion 214B that faces the central axis 126 so that at certain tilt angles of right upper jaw 214 the first portion 214A does not interfere with the radiation beam path and the edge of the face of the second portion 214B defines the aperture boundary, as shown in FIG. 2H.

In some variations, the first portion 214A may include a cap formed of, for example, steel. In some variations, each of the first jaw 212 and the second jaw 214 may include a cap or upper portion (e.g., the first portion 214A) formed of a lower attenuating material than a second portion of each of the first jaw 212 and the second jaw 214 (e.g., the second portion 214B). In use, the first jaw 212 and the second jaw 214 may be moved close enough to each other for the respective caps or upper portions to touch, nearly touch, or overlap to adjust the depth of the radiation beam 221 across the entire field.

In some variations, the first pair of tiltable jaws 210 may be arranged at an angle relative to the second pair of tiltable jaws 240 such that the first pair of tiltable jaws 210 may adjust penumbra characteristics of a radiation field 224 in a first direction and the second pair of tiltable jaws 240 may adjust penumbra characteristics of a radiation field 224 in a second direction. For example, the first pair of tiltable jaws 210 may be arranged orthogonally relative to the second pair of tiltable jaws 240 and the second direction may be orthogonal to the first direction.

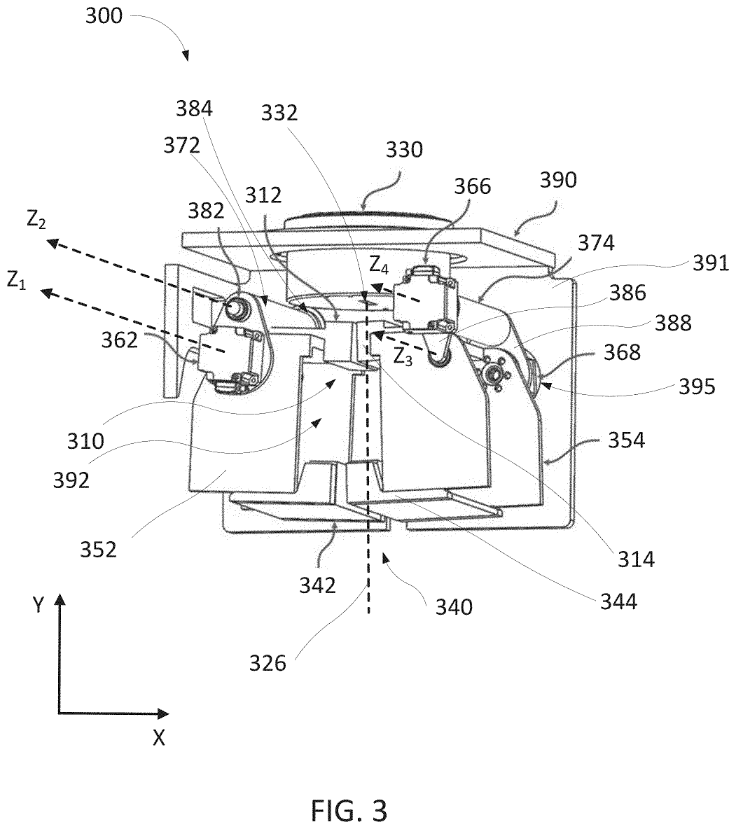

FIG. 3 is a perspective view of a radiation beam-shaping assembly 300 with a portion of a housing 390 removed to show components of the radiation beam-shaping assembly 300. The radiation beam-shaping assembly 300 may be the same or similar in structure and/or function to any of the radiation beam-shaping assemblies described herein. For example, the radiation beam-shaping assembly 300 includes a first pair of tiltable jaws 310 and a second pair of tiltable jaws 340. The first pair of tiltable jaws 310 includes a first jaw 312 and a second jaw 314. The second pair of tiltable jaws 340 includes a first jaw 342 and a second jaw 344. The first jaw 312 of the first pair of tiltable jaws 310 and the first jaw 342 of the second pair of tiltable jaws 340 are mounted to a first carriage 352. The second jaw 314 of the first pair of tiltable jaws 310 and the second jaw 344 of the second pair of tiltable jaws 340 are mounted to a second carriage 354. A primary collimator 330 may be mounted to the housing 390 of the radiation beam-shaping assembly (e.g., such that the primary collimator 330 projects through an opening of the housing 390). The primary collimator 330 may include an aperture 332 through which the primary collimator 330 may transmit a radiation beam.

The radiation beam-shaping assembly 300 includes a first shaft 372 and a second shaft 374. The first carriage 352 is coupled to the first shaft 372 via a first extension portion 382 and a second extension portion 384. The first shaft 372 has a first end and a second end. The first extension portion 382 may be coupled to the first shaft 372 near or adjacent to the first end. The second extension portion 384 may be coupled to the first shaft 372 near or adjacent to the second end. The first shaft 372 is coupled to the first extension portion 382 and the second extension portion 384 such that rotation of the first shaft 372 causes corresponding rotation of the first extension portion 382 and the second extension portion 384. For example, the first extension portion 382 and the second extension portion 384 may be mounted via screws, bolts, and/or welding to the first shaft 372. In some implementations, the first extension portion 382 and the second extension portion 384 may be integrally formed with the first shaft 372. For example, the first extension portion 382, the second extension portion 384, and the first shaft 372 may be produced from billet or from additive manufacturing (e.g., via 3D printing).

The second carriage 354 is coupled to the second shaft 374 via a third extension portion 386 and a fourth extension portion 388. The second shaft 374 has a first end and a second end. The third extension portion 386 may be coupled to the second shaft 374 near or adjacent to the first end. The fourth extension portion 388 may be coupled to the second shaft 374 near or adjacent to the second end. The second shaft 374 is coupled to the third extension portion 386 and the fourth extension portion 388 such that rotation of the second shaft 374 causes corresponding rotation of the third extension portion 386 and the fourth extension portion 388. For example, the third extension portion 386 and the fourth extension portion 388 may be mounted via screws, bolts, and/or welding to the second shaft 374. In some implementations, the third extension portion 386 and the fourth extension portion 388 may be integrally formed with the second shaft 374. For example, the third extension portion 386, the second extension portion 388, and the second shaft 374 may be produced from billet or from additive manufacturing (e.g., via 3D printing).

The radiation beam-shaping assembly 300 includes a first actuator 362, a second actuator (not shown in FIG. 3), a third actuator 366, and a fourth actuator 368. The second actuator and the third actuator 366 may be stationarily mounted to the housing 390. The first actuator 362 and the fourth actuator 368 may be configured to be rotated about the central axes of the first shaft 372 and the second shaft 374 under the control of the second actuator and the third actuator 366, respectively. The housing 390 includes openings (e.g., opening 395) shaped and sized to accommodate the range of motion of the first actuator 362 and the fourth actuator 368 under the control of the second actuator and the third actuator 366.

The first end of the first shaft 372 may be mounted to a front wall of the housing 390 (removed in FIG. 3 for visibility of internal components) and the second end of the first shaft 372 may be coupled to the second actuator. The first actuator 362 may be mounted to the first extension portion 382 such that rotation of the first shaft 372 by the second actuator causes corresponding rotation of the first actuator 362. The first actuator 362 may be coupled to the first carriage 352 via an opening in the first extension portion 382 such that the first actuator 362 is configured to rotate the first carriage 352 about a first axis Z₁ extending through the opening in the first extension portion 382. The second actuator is configured to rotate the first shaft 372 about a second axis Z₂ extending through the first shaft 372. The second axis Z₂ may be coaxial with a central axis of the first shaft 372, and the first axis Z₁ and the second axis Z₂ may be parallel to each other and perpendicular to a central axis 326 of the aperture 332 of the primary collimator 330.

The first end of the second shaft 374 may be coupled to the third actuator 366 (which may be mounted to the front wall of the housing 390 (not shown)) and the second end of the second shaft 374 may be mounted to a back wall 391 of the housing 390. The fourth actuator 368 may be mounted to the fourth extension portion 388 such that rotation of the second shaft 374 causes corresponding rotation of the fourth actuator 368. The fourth actuator 368 may be coupled to the second carriage 354 via an opening in the fourth extension portion 388 such that the fourth actuator 368 is configured to rotate the second carriage 354 about a third axis Z₃ extending through the opening in the fourth extension portion 388. The third actuator 366 is configured to rotate the second shaft 374 about a fourth axis Z₄ extending through the second shaft 374. The fourth axis Z₄ may be coaxial with a central axis of the second shaft 374, and the third axis Z₃ and the fourth axis Z₄ may be parallel to each other and perpendicular to the central axis 326 of the aperture 332 of the primary collimator 330.

Each jaw of the first pair of tiltable jaws 310 and each jaw of the second pair of tiltable jaws 340 include at least one collimation element. Each collimation element may be configured to obstruct at least a portion of a radiation beam transmitted through the aperture 332 of the primary collimator 330 when the collimation element is disposed in a path of the radiation beam. For example, each collimation element may be formed as a dense, solid body including high impedance materials (e.g., tungsten) configured to obstruct at least a portion of the radiation beam. Thus, first carriage 352 and/or the second carriage 354 may be moved (e.g., tilted, rotated, and/or laterally translated) toward and/or away from each other and a central axis of a radiation beam transmitted through the aperture 332 to adjust the size and/or shape of a first aperture between the first jaw 312 and the second jaw 314 and a second aperture between the first jaw 342 and the second jaw 344 through which the radiation beam passes. Adjustment of the first aperture and/or the second aperture may adjust the size, shape, and/or other characteristics (e.g., fluence) of the radiation field 324 by obstructing a portion of the radiation beam from passing through each collimation element of the first pair of tiltable jaws 310 and the second pair of tiltable jaws 340. Each collimation element may have any suitable shape and/or size. For example, since the first pair of tiltable jaws 310 is closer to the aperture 332 and, therefore, may interact with a smaller portion of a radiation beam projecting from the aperture 332 than the second pair of tiltable jaws 340, each collimation element of the first pair of tiltable jaws 310 may be smaller (e.g., shorter along the Z-axis and/or the X-axis) than each collimation element of the second pair of tiltable jaws 340. In some implementations, each collimation element may include a scatter shield portion extending away from a primary portion of the collimation element and away from the central axis 326. The scatter shield portion may be reduced in thickness compared to the primary portion, reducing the overall weight of the first pair of tiltable jaws 310 and the second pair of tiltable jaws 340 compared to pairs of tiltable jaws having a similar thickness throughout. Additionally, the radiation-facing surface of each of the collimation elements (e.g., of the primary portion) may be flat and form a 90° angle with the upper surface of each collimation element (e.g., the upper surface of the primary portion). In some implementations, each collimation element may include one or more curved surfaces.

Furthermore, the radiation beam-shaping assembly 300 may define an interior space 392 within which a multi-leaf collimator assembly (not shown) may be disposed such that the multi-leaf collimator assembly may further shape the radiation beam transmitted from the primary collimator 330. The multi-leaf collimator assembly may be disposed, for example, between the first pair of tiltable jaws 310 and the second pair of tiltable jaws 340. In some variations, for example, the multi-leaf collimator may be configured to shape the radiation beam in a first direction and the first pair of tiltable jaws 310 and the second pair of tiltable jaws 340 may be configured to shape the radiation beam in a second direction perpendicular to the first direction.

The first actuator 362, the second actuator, the third actuator 366, and the fourth actuator 368 may each be or include any suitable actuator configured to control the orientation and position of the first carriage 352 or the second carriage 354, respectively, as described herein. In some implementations, the first jaw 312 and the second jaw 314 of the pair of upper jaws 310 may each have a mass of, for example, between about 8 kg and about 12 kg (e.g., about 10 kg). The first jaw 342 and the second jaw 344 of the pair of lower jaws 340 may each have a mass of, for example, between about 13 kg and about 17 kg (e.g., about 15 kg). Therefore, the first actuator 362, the second actuator, the third actuator 366, and the fourth actuator 368 may be configured to apply sufficient output torque and have a sufficient moment stiffness to control the rotation of each of the jaws of the pair of upper jaws 310 and each of the jaws of the pair of lower jaws 340. For example, the first actuator 362, the second actuator, the third actuator 366, and the fourth actuator 368 may each be rotary actuators. In some implementations, the first actuator 362, the second actuator, the third actuator 366, and the fourth actuator 368 may be configured to apply an output torque of between about 20 N·m and 28 N·m. In some implementations, each of the first actuator 362, the second actuator, the third actuator 366, and the fourth actuator 368 may each have a moment stiffness of about 8×10⁴ N·m/rad. In some implementations, the second actuator may be configured to rotate the first shaft 372 at a rotational speed of between about 50 and about 60 revolutions per minute (RPM). In some implementations, the third actuator 366 may be configured to rotate the second shaft 374 at a rotational speed of between about 50 and about 60 RPM. In some implementations, the first actuator 362 may be configured to rotate the first carriage 352 about the first axis Z₁ at a rotational speed of between about 50 and about 60 RPM and the fourth actuator 368 may be configured to rotate the second carriage 354 about the third axis Z₃ at a rotational speed of between about 50 and about 60 RPM. In some variations, each of the first actuator 362, the second actuator, the third actuator 366, and the fourth actuator 368 may be configured to rotate the first pair of tiltable jaws 310 and the second pair of tiltable jaws 340 sufficiently quickly such that the orientation and position of each jaw may be adjusted for each firing position of a rotatable gantry to which the radiation beam-shaping assembly 300 is coupled (e.g., about every 10 ms).

Although the radiation beam-shaping assembly 300 is shown in FIG. 3 as being arranged with the first actuator 362 and the third actuator 366 on a first side of the radiation beam-shaping assembly 300 and the second actuator and the fourth actuator 368 on a second side of the radiation beam-shaping assembly 300, in some implementations the first actuator 362 and the fourth actuator 368 may be arranged on a first side of the housing and the second actuator and the third actuator 366 may be arranged on a second side of the housing. In such an implementation, the first extension member 382 and the fourth extension member 388 may be disposed such that the first extension member 382 and the fourth extension member 388 mirror each other about the central axis 326 of the aperture 332 and the second extension member 384 and the third extension member 386 may be disposed such that the second extension member 384 and the third extension member 386 mirror each other about the central axis 326 of the aperture 332.

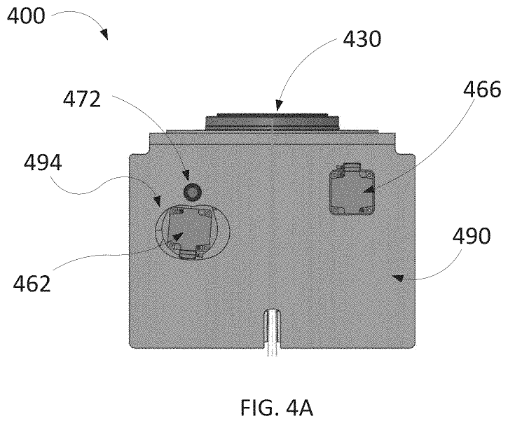

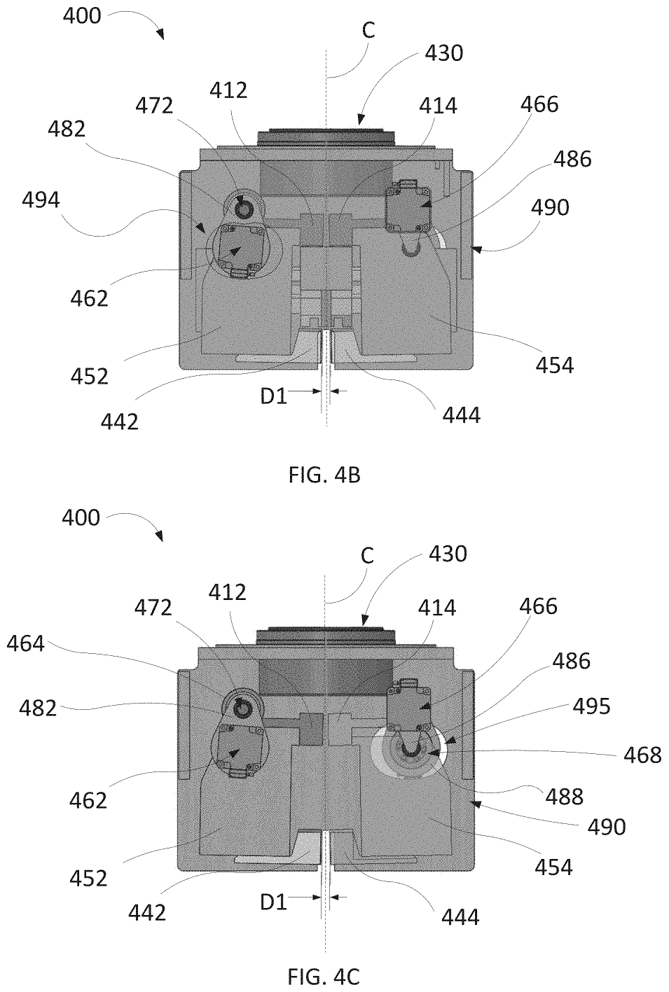

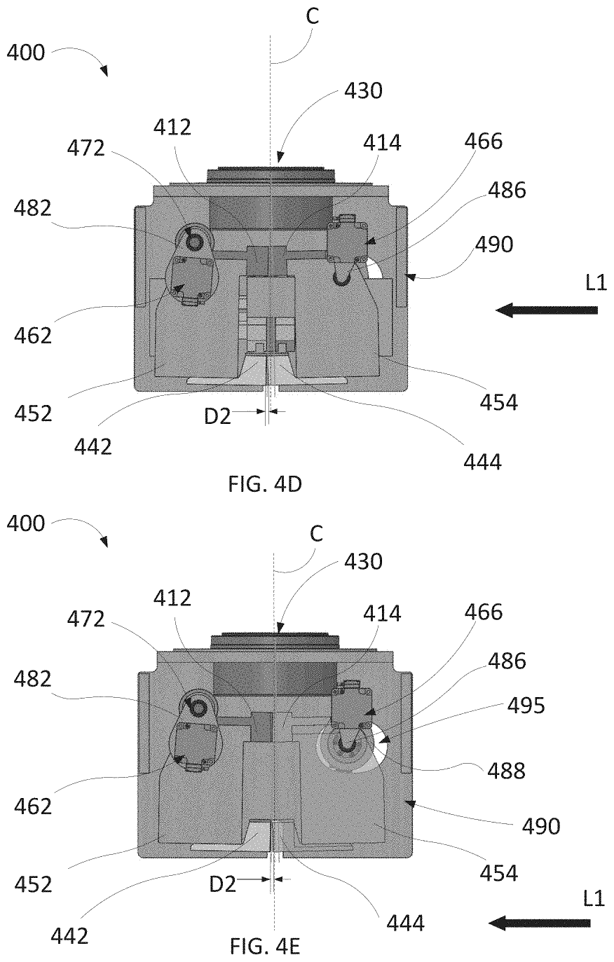

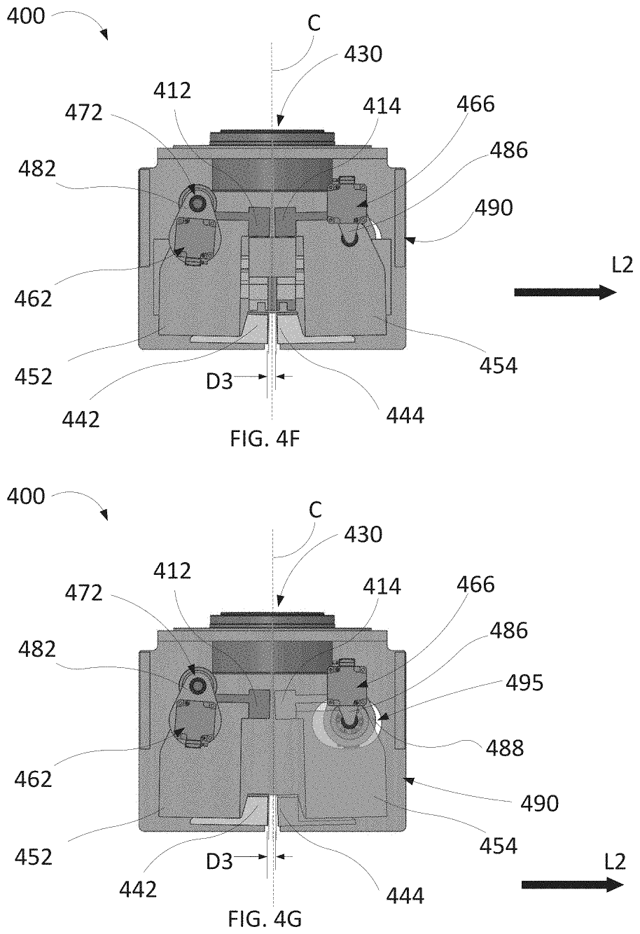

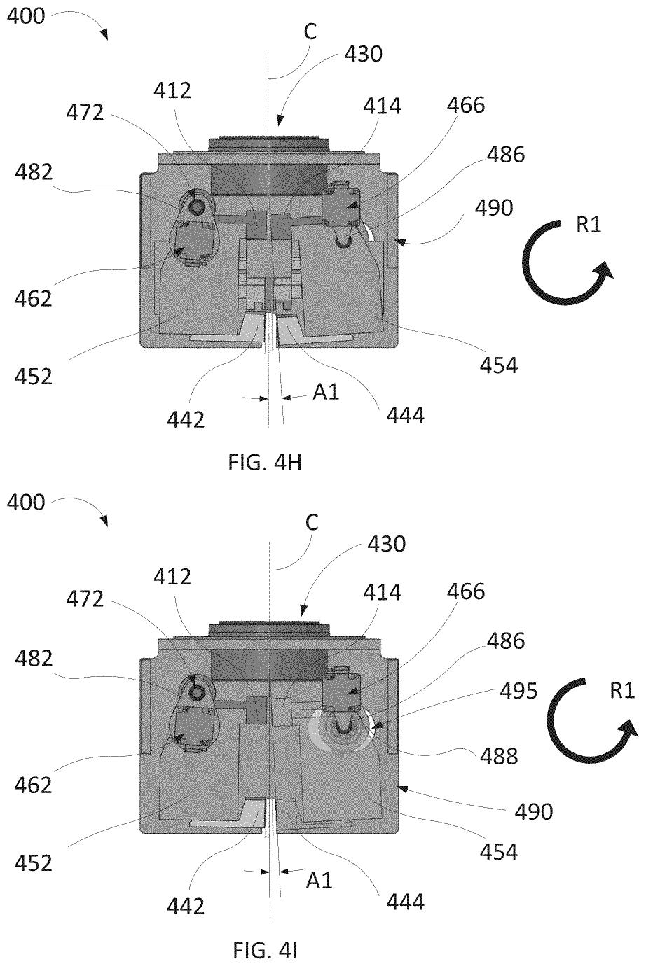

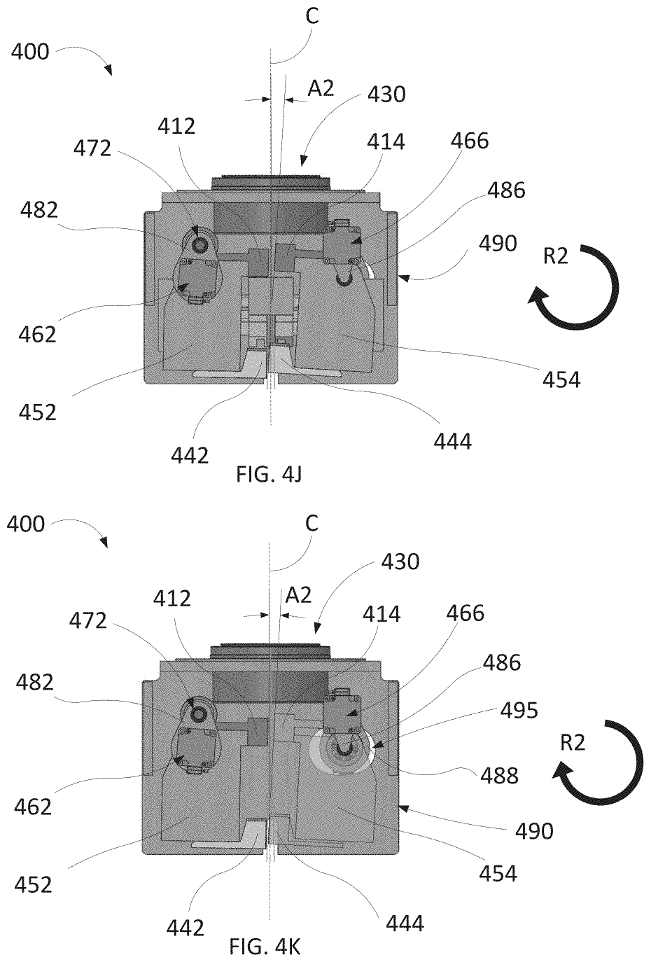

FIGS. 4A-4K are various illustrations of a radiation beam-shaping assembly 400 in a variety of configurations. The radiation beam-shaping assembly 400 may be the same or similar in structure and/or function to any of the radiation beam-shaping assemblies described herein such as, for example, the radiation beam-shaping assembly 300.

FIG. 4A is a front view of the radiation beam-shaping assembly 400. The radiation beam-shaping assembly 400 includes a housing 490, a first actuator 462, a second actuator 464 (shown in FIG. 4C), a third actuator 466, and a fourth actuator 468 (shown in FIG. 4C). The radiation beam-shaping assembly 400 also includes a first shaft 472 having a first end mounted to the housing 490 and a second end mounted to the second actuator 464, and a second shaft (not shown) having a first end mounted to the third actuator 466 and a second end mounted to the housing. The housing 490 may be configured to support a primary collimator 430. The housing 490 may define a first opening 494 sized and shaped such that the first actuator 462 may be rotated under control of the second actuator 464 along a path (e.g., a linear or an arcuate path) within the first opening 494. The housing 490 may define a second opening 495 (shown in FIG. 4C) such that the fourth actuator 468 may be rotated under control of the third actuator 466 along a path (e.g., a linear or an arcuate path) within the second opening 495.

FIGS. 4B and 4C are front views of the radiation beam-shaping assembly 400 in a first configuration. FIG. 4B shows the radiation beam-shaping assembly 400 with a front wall of the housing 490 removed. FIG. 4C shows the radiation beam-shaping assembly 400 with the front wall removed and with a second carriage 454 shown as being transparent. As shown in FIGS. 4B and 4C, the radiation beam-shaping assembly 400 includes a first carriage 452 and the second carriage 454. The first carriage 452 is coupled to the first shaft 472 via a first extension member 482 and a second extension member (not shown). The second carriage 454 is coupled to the second shaft 474 via a third extension member 486 and a fourth extension member 488. The radiation beam-shaping assembly 400 includes a first pair of tiltable jaws including a first jaw 412 and a second jaw 414 and a second pair of tiltable jaws including a first jaw 442 and a second jaw 444. The first jaw 412 and the first jaw 442 may be mounted to the first carriage 452 and the second jaw 414 and the second jaw 444 may be mounted to the second carriage 454. The first jaw 442 and the second jaw 444 are separated by a first distance D1 in the first configuration of the radiation beam-shaping assembly 400 and positioned on opposite sides of a central axis C of an opening of the primary collimator 430 through which a radiation beam may be transmitted. The first distance D1 may correspond to a radiation field including a primary portion and a secondary portion each having a first size.

The first actuator 462 is coupled to the first carriage 452 via an opening in the first extension member 482. The first extension member 482 is coupled to the first shaft 472 such that rotation of the first shaft 472 causes corresponding rotation of the first extension member 482. The first actuator 462 is configured to control the relative motion and/or position between the first carriage 452 and the first extension member 482. The first actuator 462 may rotate the first carriage 452 clockwise and/or counterclockwise relative to the first extension member 482 and the first shaft 472. The first actuator 462 may also hold the first carriage 452 stationary relative to the first extension member 482 and the first shaft 472.

The fourth actuator 468 is coupled to the second carriage 454 via an opening in the fourth extension member 488. The fourth extension member 488 is coupled to the second shaft such that rotation of the second shaft causes corresponding rotation of the fourth extension member 488. The fourth actuator 488 is configured to control the relative motion and/or position between the second carriage 454 and the fourth extension member 488. The fourth actuator 488 may rotate the second carriage 454 clockwise and/or counterclockwise relative to the fourth extension member 488 and the second shaft. The fourth actuator 488 may also hold the second carriage 454 stationary relative to the fourth extension member and the second shaft.

FIGS. 4D and 4E are front views of the radiation beam-shaping assembly 400 in a second configuration where the aperture defined by the space between the first and second jaws of each of the pair of jaws has been reduced. FIG. 4D shows the radiation beam-shaping assembly 400 with a front wall of the housing 490 removed. FIG. 4E shows the radiation beam-shaping assembly 400 with the front wall removed and with the second carriage 454 shown in transparent. As shown in FIGS. 4D and 4E, the second carriage 454 has been translated laterally in the direction of arrow L1 toward the central axis C and the first carriage 452 through a combined motion of the third actuator 466 and the fourth actuator 468. The distance between the first jaw 442 and the second jaw 444 has been reduced from the first distance D1 to a second distance D2. The second distance D2 may correspond to a radiation field having a primary portion having a second size smaller than the first size. The third actuator 466 rotated the second carriage 454 in a first direction (e.g., clockwise) and the fourth actuator 468 counterrotated the second carriage 454 in a second direction (e.g., counter-clockwise) such that the combined motion of the second carriage 454 is a linear lateral translation in the direction of arrow L1 toward the first carriage 452. As shown, the fourth actuator 468 was moved within the second opening 495 toward the first carriage 452 under the control of the third actuator 466. Alternatively or additionally, the fourth actuator 468 may not counterrotate the second carriage 452 in the second direction and/or the lateral motion may be along a curvilinear or arcuate path.

FIGS. 4F and 4G are front views of the radiation beam-shaping assembly 400 in a third configuration where the aperture defined by the space between the first and second jaws of each of the pair of jaws has been increased. FIG. 4F shows the radiation beam-shaping assembly 400 with a front wall of the housing 490 removed. FIG. 4G shows the radiation beam-shaping assembly 400 with the front wall removed and with the second carriage 454 shown in transparent. As shown in FIGS. 4F and 4G, the second carriage 454 has been translated laterally in the direction of arrow L2 away from the first carriage 452 and the central axis C through a combined motion of the third actuator 466 and the fourth actuator 468. The distance between the first jaw 442 and the second jaw 444 has been increased from the second distance D2 to a third distance D3. The third distance D3 may correspond to a radiation field having a primary portion having a third size greater than the second size. The third size may be greater than, less than, or equal to the first size. The primary portion of the radiation field may have the same fluence regardless of the size of the radiation field. The third actuator 466 rotated the second carriage 454 in a second direction (e.g., counter-clockwise) and the fourth actuator 468 counterrotated the second carriage 454 in a first direction (e.g., clockwise) such that the combined motion of the second carriage 454 is a linear lateral translation in the direction of arrow L2 away the first carriage 452. As shown, the fourth actuator 468 was moved within the second opening 495 away from the first carriage 452 under the control of the third actuator 466. Alternatively or additionally, the fourth actuator 468 may not counterrotate the second carriage 452 in the second direction and/or the lateral motion may be along a curvilinear or arcuate path.

FIGS. 4H and 4I are front views of the radiation beam-shaping assembly 400 in a fourth configuration where the shape of the aperture defined by the space between the first and second jaws of each of the pair of jaws has been changed by adjusting a tilt angle between a radiation-facing surface of the jaws of the second carriage 454 and the central axis C of the opening of the primary collimator 430 through which a radiation beam may be transmitted. FIG. 4H shows the radiation beam-shaping assembly 400 with a front wall of the housing 490 removed. FIG. 4I shows the radiation beam-shaping assembly 400 with the front wall removed and with the second carriage 454 shown in transparent. As shown in FIGS. 4H and 4I, the second carriage 454 has been tilted toward the first carriage 452 in the rotational direction of arrow R1 such that the first jaw 414 is rotated toward the first carriage 452 and the second jaw 444 is rotated away from the first carriage 452. The third actuator 466 maintains the second shaft in the same orientation as in the third configuration of the radiation beam-shaping assembly 400, and the fourth actuator 468 rotated the second carriage 454 relative to the third actuator 466, the second shaft, and the fourth extension member 488. As a result, the second carriage 454 is rotated to an angle A1 relative to the central axis C of the opening of the primary collimator 430. The angle A1 may be, for example, about 5 degrees, about 2 degrees, about 3.11 degrees, about 0.93 degrees, about 0.62 degrees, or any other suitable angle. Adjusting the angle A1 may adjust a size of a secondary or penumbra portion extending from a primary portion in a region of a radiation field corresponding to the second carriage 454 (e.g., on a side of the radiation field disposed below the second carriage 454 relative to the primary collimator 430). For example, the distance an edge of the secondary portion extends relative to an edge of the primary portion may correspond to the size of the angle A1. If the angle A1 increases, the distance between the edge of the secondary portion and the edge of the primary portion may increase accordingly (e.g., the size of the penumbra region may increase). Additionally or alternatively, the angle A1 may correspond to a sharpness of a boundary of the primary region (e.g., a knee) or the secondary region such that the smaller the angle A1, the sharper the boundary of the primary or the secondary region (e.g., the size of the penumbra region may decrease).

FIGS. 4J and 4K are front views of the radiation beam-shaping assembly 400 in a fifth configuration where the shape of the aperture defined by the space between the first and second jaws of each of the pair of jaws has been changed by adjusting the tilt angle between the radiation-facing surface of the jaws of the second carriage 454 and the central axis C of the opening of the primary collimator 430 through which the radiation beam may be transmitted. FIG. 4J shows the radiation beam-shaping assembly 400 with a front wall of the housing 490 removed. FIG. 4K shows the radiation beam-shaping assembly 400 with the front wall removed and with the second carriage 454 shown in transparent. As shown in FIGS. 4J and 4K, the second carriage 454 has been tilted away from the first carriage 452 in the rotational direction of arrow R2 such that the second jaw 414 is rotated away the first carriage 452 and the second jaw 444 is rotated toward the first carriage 452. The third actuator 466 maintains the second shaft in the same orientation as in the third and fourth configurations of the radiation beam-shaping assembly 400, and the fourth actuator 468 rotated the second carriage 454 relative to the third actuator 466, the second shaft, and the fourth extension member 488. As a result, the second carriage 454 is rotated to an angle A2 relative to the central axis C of the opening of the primary collimator 430. The angle A2 may be, for example, opposite and equal the first angle A1. The angle A2 may correspond to size of a secondary or penumbra portion extending from a primary portion in a region of a radiation field corresponding to the second carriage 454 (e.g., on a side of the radiation field disposed below the second carriage 454 relative to the primary collimator 430). For example, the distance an edge of the secondary portion extends relative to an edge of the primary portion may correspond to the size of the angle A2. If the angle A2 increases, the distance between the edge of the secondary portion and the edge of the primary portion may increase accordingly (e.g., the size of the penumbra region may increase). Additionally or alternatively, the angle A2 may correspond to the sharpness of an edge of a primary or secondary portion of a radiation field. For example, the smaller the angle A2, the more sharp an edge of the primary portion of the radiation field and the smaller the distance between an edge of the primary and an edge of the second portion on a side of the radiation field disposed below the second carriage 454 relative to the primary collimator 430 (e.g., the size of the penumbra region may decrease).

Additionally, transitioning the second carriage 454 to the angle A2 may cause an increase in radiation dose delivery. When the first carriage 452 and the second carriage 454 are arranged (e.g., tilted) relative to each other such that the aperture formed by the first upper jaw 412, the first lower jaw 442, the second upper jaw 414, and the second lower jaw 444 is tapered toward the patient (e.g., funnel-shaped, where the width of the aperture formed by the upper jaws is greater than the width of the aperture formed by the lower jaws), the radiation beam-shaping assembly 400 can help to focus the radiation field or the primary region of the radiation field. The funnel-shaped arrangement of jaws 412, 414, 442, 444 formed by tilting the jaws at an angle A2 may help increase the fluence through the beam-shaping assembly 400 by directing any scattered photons “forward” or through the aperture towards the patient. That is, tilting the jaws may help increase the number of photons that are “scattered forward” and reduce the number of photons that are scattered in other directions, e.g., away from the patient, diffuse or dispersed directions.

Although only the second carriage 454 of the radiation beam-shaping assembly 400 is shown translating and rotating (e.g., tilting) in FIGS. 4A-4K, in some variations the first carriage 452 may also translate and rotate relative to the second carriage 454 and/or the central axis C of the opening of the primary collimator 430 through which a radiation beam may be transmitted. The first carriage 452 and the second carriage 454 may be translated and rotated independently and in concert with each other (e.g., oppositely about the central axis C of the opening of the primary collimator 430).

In some implementations, a radiation beam-shaping assembly, such as any of the radiation beam-shaping assemblies described herein, may comprise or be coupled to a controller in communication with a group of actuators of the radiation beam-shaping assembly. The controller may comprise one or more processors and one or more machine-readable memories in communication with the one or more processors. The controller may be connected to each actuator from the group of actuators by wired or wireless communication channels. The controller may be located in the same room or bunker as the radiation beam-shaping assembly, or may be located in a different room or bunker from the radiation beam-shaping assembly. In some variations, the controller and the radiation beam-shaping assembly may both be located on a gantry of a radiation therapy system. The controller may be configured to coordinate movement of each jaw of the radiation beam-shaping assembly by controlling one or more actuators corresponding to each jaw. The controller may also be configured to detect the position of each jaw of the radiation beam-shaping assembly. In some implementations, the controller may be configured to coordinate the movement of a couch with the rotation of the gantry (e.g., speed), activate one or more radiation sources, open or close other collimator leaves/jaws, detect the position of other the collimator leaves/jaws, detect positron emission paths, detect MV radiation applied to the patient, compute delivered dose based on detected MV radiation data, store treatment plan data, and/or store anatomical data from other imaging modalities including, but not limited to, MRI, CT, ultrasound, flat detectors, PET, ion chambers, etc. The controller may use such detected data and/or information to update radiation delivery, for example, by adjusting the rotation speed of the ring, rotating and/or laterally translating one or more jaws of the radiation beam-shaping assembly, opening or closing certain leaves of a multi-leaf collimator assembly disposed under the therapeutic radiation source, and/or by adjusting the timing of the therapeutic radiation pulses.

In some implementations, a radiation beam-shaping assembly such as any of the radiation beam-shaping assemblies described herein may be included in any suitable radiation therapy system. The radiation beam-shaping assembly may be configured to rotate and/or translate jaws of the radiation beam-shaping assembly between various configurations such that the jaws are in a particular position relative to a patient target region and a source of radiation depending on the location of the radiation beam-shaping assembly and the source of radiation relative to the patient target region. For example, the jaws of the radiation beam-shaping assembly may be capable of changing to different configurations for each firing station, couch beam station, and/or shuttle pass. In some implementations, the radiation beam-shaping assembly may be configured to begin transitioning the jaws to a configuration associated with a firing station, a couch beam station, and/or a shuttle pass prior to the radiation beam-shaping assembly being positioned relative to the particular firing station, couch beam station, and/or shuttle pass, reducing radiation therapy treatment time. For example, a radiation beam-shaping assembly may have first configuration for first couch beam station, and then transition to a second configuration for a second couch beam station. Alternatively or additionally, a radiation beam-shaping assembly may have first configuration for first shuttle pass, and then transition to a second configuration for a second shuttle pass. Optionally, a radiation beam-shaping assembly may change configurations for different firing stations in a given couch beam station and shuttle pass.

In some implementations, a radiation beam-shaping assembly such as any of the radiation beam-shaping assemblies described herein may be included in a radiation therapy system comprising a continuously-rotating gantry. The gantry may be configured to rotate 360 degrees or more in one or more directions (e.g., capable of rotating 360 degrees or more counterclockwise and/or rotating 360 degrees or more clockwise). A continuously-rotating gantry may receive its rotational force from a traditional motor and coupled drive system or from an integrated rotor and stator design. To reduce latencies from the time a lesion or target region is located to therapeutic radiation delivery, the system may rotate a therapeutic radiation source, the radiation beam-shaping assembly, and other delivery hardware at much higher speeds than traditional radiotherapy systems. A radiation therapy system may comprise rotor and stator elements that are integrated into the same structure that supports the bearings, which may help the gantry rotate several tons of hardware continuously (e.g., 360 degrees) at speeds up to about 70 RPM (e.g., at least about 50 RPM, about 60 RPM, etc.). The radiation beam-shaping assembly may include a group of actuators as described herein, which may be configured to rotate and/or translate jaws of the radiation beam-shaping assembly at rates of speed corresponding to the rotational speed of the gantry. Thus, actuators included in a system including a gantry that is capable of rotating at speeds from about 10 RPM to about 70 RPM may each be configured to rotate a jaw at a speed from about 10 RPM to about 70 RPM.

In some implementations, a radiation beam-shaping assembly, such as any of the radiation beam-shaping assemblies described herein, may include one or more iris diaphragms (e.g., one, two, three or more iris diaphragms) as an alternative to or in addition to the one or more pairs of jaws described as being included in the radiation beam-shaping assemblies described herein. The iris diaphragms may be similar in structure and/or function to the pairs of jaws described herein. For example, an iris diaphragm may include a set of blades that are each formed as a dense, solid body including attenuating materials (e.g., tungsten, tungsten alloy, lead, lead alloy, iron, iron alloy, steel alloy, or any other suitable alloy and/or high-impedance materials) configured to obstruct a radiation beam. The blades may be arranged relative to one another in a radial pattern to define an opening through which a portion of a radiation beam (e.g., radiation beam 121 or radiation beam 221) may pass. Each blade may be articulated or rotated about a distinct axis such that each blade may be rotated toward and away from a central axis (e.g., central axis 126 or central axis 226) extending between a source of radiation (e.g., source of radiation 120 or source of radiation 220) and a patient target region (e.g., between a primary collimator such as primary collimator 130 or primary collimator 230 and a patient target region). Each distinct axis associated with a blade may be parallel to the remaining axes associated with the remaining blades, and the axes associated with the blades may be parallel to the central axis extending between the source of radiation and the patient target region. As each blade is rotated about their respective axis toward and away from the central axis, the size and the shape of the opening may be adjusted to change one or more characteristics of a radiation field (e.g., radiation field 124 or radiation field 224) such as the size, shape, outer boundary, sharpness, fluence, and/or energy delivery depth or the radiation field (e.g., of a primary and/or secondary region of the radiation field). In some implementations, each blade may be associated with a distinct actuator such that the blade may be independently actuated (e.g., under the control of one or more controllers as described herein) relative to the remaining blades of the iris diaphragm. In some implementations, all of the blades or subsets of the blades of the iris diaphragm may be moved in unison under the control of one or more common actuators (e.g., under the control of one or more controllers as described herein).

In some variations, the opening collectively defined by the blades may be any suitable shape. For example, in one or more configurations of the blades, the opening may be circular, ovoid, conical, curvilinear, hexagonal, pentagonal, square, or rectangular. In some variations, in one or more configurations of the blades, the opening may be asymmetrical or symmetrical. In some variations, the blades of the iris diaphragm may have any suitable shape. For example, the blades may be triangular or curvilinear. In some implementations, the blades may be configured to partially overlap in one or more configurations of the blades. In some implementations, the blades may be configured to rotate within a common plane also containing the opening defined by the blades. In some implementations, the edge of each blade defining a portion of the opening may be straight or curved. The iris diaphragm may include any suitable number of blades (e.g., two, three, four, five, six, seven, eight, nine, ten, or more blades).

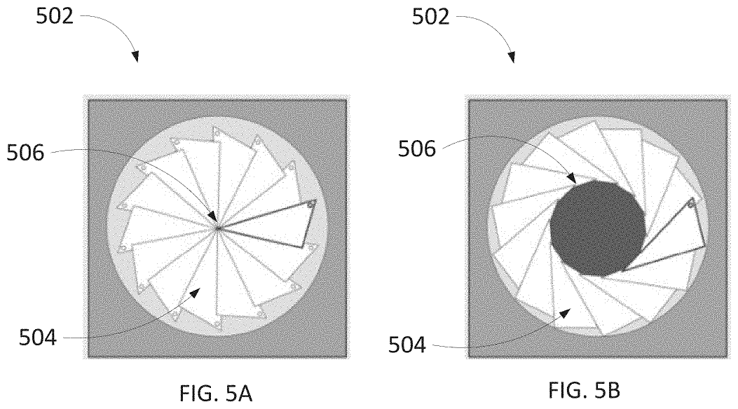

As an example, FIGS. 5A and 5B are schematic illustrations of an iris diaphragm 502 including a set of blades 504 in a first configuration and a second configuration, respectively. As shown in FIG. 5A, the blades 504 may be disposed in a first configuration in which an opening 506 collectively defined by the blades 504 has a first size. The blades 504 may be rotated to a second configuration (shown in FIG. 5B) in which the opening 506 collectively defined by the blades 504 has a second size greater than the first size. As a result, the radiation field generated by projecting radiation from a source of radiation through the opening 506 in the second configuration of the iris diaphragm 502 may have different characteristics (e.g., a greater size) compared to the characteristics of a radiation field associated with the first configuration.

In some implementations, two or more iris diaphragms may be disposed along the central axis (e.g., in a stacked arrangement) such that the configuration of the blades of each iris diaphragm may be adjusted to change one or more characteristics of a radiation field. For example, one or more blades of a first iris diaphragm disposed closer to the source of radiation than a second diaphragm may be disposed such that the blades are capable of obstructing a first portion of a radiation beam at the first distance from the source of radiation, and one or more blades of the second iris diaphragm may be disposed such that the blades are capable of obstructing a second portion of the radiation beam at a second distance from the source of radiation. In some implementations, the blades of each iris diaphragm may be independently actuated relative to the blades of other iris diaphragms of the radiation beam-shaping assembly. In some implementations, the blades of each iris diaphragm may be configured to move in concert with one another (e.g., each iris diaphragm may be configured to define openings of the same shape and size as the remaining iris diaphragm(s) throughout operation of the radiation beam-shaping assembly).

In some implementations, the blades of an iris diaphragm that is disposed closer to the source of radiation may be formed of lesser attenuating materials compared to blades of an iris diaphragm that is disposed farther from the source of radiation. In some implementations, one or more of the iris diaphragms may be associated with an actuator configured to translate the iris diaphragm within a plane perpendicular to the central axis extending between the source of radiation and the patient target region. In some implementations, the distance between each iris diaphragm and an adjacent iris diaphragm may be any suitable distance (e.g., a distance greater than is required for mechanical clearance between the adjacent iris diaphragms) such that a particular characteristic of a radiation field may be achieved. In some implementations, each iris diaphragm may be configured to be translated toward or away from the source of radiation (e.g., along a central axis extending between the source of radiation and the patient target region).

In some implementations, a radiation beam-shaping assembly, such as any of the radiation beam-shaping assemblies described herein, may include one or more independently moveable plates (e.g., one, two, three, four, five or more independently moveable plates) as an alternative to or in addition to the one or more pairs of jaws and/or to the one or more iris diaphragms described as being included in the radiation beam-shaping assemblies described herein. For example, independently articulated plates may be disposed at different distances from a source of radiation (e.g., stacked) and configured to be translated or rotated relative to a central axis extending between the source of radiation and a patient target region (e.g., into and out of a beam path to shape a radiation beam generated by the source of radiation). Each independently moveable plate may define one or more openings (e.g., holes). Each hole may be any suitable shape, such as circular, ovoid, curvilinear, or rectangular. Each plate may be associated with a distinct actuator such that each plate may be separately controlled by a controller to be transitioned between various orientations and/or positions (e.g., via being rotated and/or translated). For example, the orientation and/or position of each plate may be adjusted similarly as described above with respect to the jaws (e.g., jaw 112, jaw 212, and/or jaw 242).

While various embodiments have been described above, it should be understood that they have been presented by way of example only, and not limitation. Where methods described above indicate certain events occurring in certain order, the ordering of certain events may be modified. Additionally, certain of the events may be performed concurrently in a parallel process when possible, as well as performed sequentially as described above.

Where schematics and/or embodiments described above indicate certain components arranged in certain orientations or positions, the arrangement of components may be modified. While the embodiments have been particularly shown and described, it will be understood that various changes in form and details may be made. Any portion of the apparatus and/or methods described herein may be combined in any combination, except mutually exclusive combinations. The embodiments described herein may include various combinations and/or sub-combinations of the functions, components, and/or features of the different embodiments described.

1. A radiation beam-shaping assembly comprising:
a pair of tiltable jaws, wherein each jaw of the pair of tiltable jaws is laterally movable relative to a radiation beam path.

2. The radiation beam-shaping assembly of claim 1, wherein a first jaw of the pair of tiltable jaws is movable in concert with a second jaw of the pair of tiltable jaws.

3. The radiation beam-shaping assembly of claim 1, wherein lateral movement of each jaw of the pair of tiltable upper jaws relative to the radiation beam path adjusts the size of an aperture defined between the pair of tiltable upper jaws such that a radiation beam field is adjusted.

4. The radiation beam-shaping assembly of claim 1, wherein a first actuator and a second actuator are coupled to a first jaw of the pair of tiltable jaws, a lateral movement of the first jaw based, at least in part, on a combined rotation of the first jaw by the first actuator and counterrotation of the first jaw by the second actuator.

5. The radiation beam-shaping assembly of claim 1, wherein the pair of tiltable jaws is a pair of tiltable upper jaws, further comprising:
a pair of tiltable lower jaws, wherein each jaw of the pair of tiltable lower jaws is laterally movable relative to a radiation beam path.

6. The radiation beam-shaping assembly of claim 5, wherein a first jaw of the pair of tiltable upper jaws is movable in concert with a first jaw of the pair of tiltable lower jaws.

7. The radiation beam-shaping assembly of claim 6, wherein a second jaw of the pair of tiltable upper jaws is movable in concert with a second jaw of the pair of tiltable lower jaws.

8. The radiation beam-shaping assembly of claim 5, wherein a first jaw of the pair of tiltable upper jaws and a first jaw of the pair of tiltable lower jaws are mounted to a first carriage and a second jaw of the pair of tiltable upper jaws and a second jaw of the pair of tiltable lower jaws are rotatably mounted to a second carriage.

9. The radiation beam-shaping assembly of claim 5, further comprising:
a third pair of tiltable jaws, wherein each jaw of the third pair of tiltable jaws is laterally movable relative to a radiation beam path.

10. The radiation beam-shaping assembly of claim 5, further comprising:
a multi-leaf collimator assembly located between the pair of tiltable upper jaws and the pair of tiltable lower jaws.

11. The radiation beam-shaping assembly of claim 1, wherein the lateral movement of each jaw of the pair of tiltable jaws is linear.

12. The radiation beam-shaping assembly of claim 1, wherein the lateral movement of each jaw of the pair of tiltable jaws is curvilinear.

13. The radiation beam-shaping assembly of claim 1, wherein the pair of tiltable jaws is a pair of tiltable upper jaws, further comprising:
a pair of tiltable lower jaws, wherein each jaw of the pair of tiltable lower jaws is fixed relative to a radiation beam path.

14. The radiation beam-shaping assembly of claim 1, wherein each jaw of the pair of tiltable jaws includes an upper portion and a lower portion, the upper portion formed of a lower attenuating material than the lower portion.

15. The radiation beam-shaping assembly of claim 14, wherein each of the upper portion and the lower portion includes a surface facing a centerline of the radiation beam path, the surface of the upper portion being angled relative to the surface of the lower portion.
